(19) United States
(12) Patent Application Publication
(10) Pub. No.: US 2023/0005647 A1
(43) Pub. Date: Jan. 5, 2023

(54) BIOCOMPATIBLE OILY FERROFLUID AND PREPARATION PROCESS

(71) Applicants: UNIVERSITE DE BORDEAUX, Bordeaux (FR); INSTITUT POLYTECHNIQUE DE BORDEAUX, Talence (FR); CENTRE NATIONAL DE LA RECHERCHE SCIENTIFIQUE, Paris (FR); CENTRE HOSPITALIER UNIVERSITAIRE DE BORDEAUX, Talence (FR)

(72) Inventors: Stéphane MORNET, SAINT VINCENT DE PAUL (FR); Clément VECCO-GARDA, CHASSIEU (FR); Sylvie CRAUSTE-MANCIET, BORDEAUX (FR); Olivier SANDRE, PESSAC (FR); Franck COUILLAUD, PESSAC (FR); Pauline JEANJEAN, MERIGNAC (FR); Coralie GENEVOIS, BORDEAUX (FR)

(73) Assignees: UNIVERSITE DE BORDEAUX, Bordeaux (FR); INSTITUT POLYTECHNIQUE DE BORDEAUX, Talence (FR); CENTRE NATIONAL DE LA RECHERCHE SCIENTIFIQUE, Paris (FR); CENTRE HOSPITALIER UNIVERSITAIRE DE BORDEAUX, Talence (FR)

(21) Appl. No.: 17/778,941

(22) PCT Filed: Nov. 26, 2020
(86) PCT No.: PCT/FR2020/052184

§ 371 Date: May 23, 2022

(30) Foreign Application Priority Data
Nov. 26, 2019 (FR) FR1913249

(51) Int. Cl. H01F 1/44; A61K 49/18

(52) U.S. Cl. CPC H01F 1/445; A61K 49/1839; B82Y 5/00

(57) ABSTRACT

The invention concerns a biocompatible oily ferrofluid comprising iron-oxide based magnetic nanoparticles and an oil phase comprising at least one fatty acid ester, characterized in that said magnetic nanoparticles are surface functionalized by molecules of one or more phospholipids, and in particular a biocompatible oily ferrofluid comprising iron-oxide based magnetic nanoparticles and an oil phase comprising at least one fatty acid ester, said iron-oxide based magnetic nanoparticles forming a colloidal dispersion in said oil phase from a temperature belonging to the range from 20 to 80° C., characterized in that said magnetic nanoparticles are surface functionalized by molecules of one or more phospholipids which do not completely cover the surface of the iron-oxide based magnetic nanoparticles, which in particular ensure a coverage rate of the surface of the iron-oxide based magnetic nanoparticles such that the fatty acid ester(s) present in the oil phase have access to the surface of the iron-oxide based magnetic nanoparticles. The invention also concerns the process for preparing such a biocompatible oily ferrofluid and its use as a contrast agent for magnetic resonance imaging or in the context of a cancer treatment by hyperthermia. Finally, the invention concerns a nanoemulsion comprising such a biocompatible oily ferrofluid.

The invention concerns a biocompatible oily ferrofluid comprising magnetic iron-oxide based magnetic nanoparticles dispersed in an oil phase comprising at least one fatty acid ester, the nanoparticles being surface functionalized by one or more phospholipids. The invention also concerns the process for preparing such a biocompatible oily ferrofluid and its use as a contrast agent or in a cancer treatment by magnetic-induced hyperthermia. Finally, the invention concerns a nanoemulsion comprising such a biocompatible oily ferrofluid.

A ferrofluid is made up of a kinetically stable dispersion of superparamagnetic nanoparticles in a carrier liquid which may be an aqueous or organic solvent, or an oil. A ferrofluid becomes magnetic when a static external magnetic field is applied. A ferrofluid is able to move or deform under the action of a magnetic field. In the context of use in magnetically-induced hyperthermia, the magnetic particles are used as a heating mediator under the effect of an alternating external magnetic field.

Depending on the use of these ferrofluids, the magnetic particles are dispersed in aqueous medium, in oily medium or in an emulsion.

Currently, aqueous ferrofluids are especially known for use as contrast agents in MRI and in the therapy of solid tumours by magnetic-induced hyperthermia. For example, in the context of the NanoTherm® therapy developed by MagForce AG®, iron oxide nanoparticles in aqueous suspension are injected into the tumour and agglomerate in the target tissue. Nanoparticles generate a release of heat under the effect of an external magnetic field applied, which induces the destruction of tumour cells. This treatment can be applied as a complement to chemotherapy or radiation therapy.

An effective ferrofluid must make it possible to destroy a large tumour volume in a short magnetic induction treatment time and for a low concentration of nanoparticles. However, the calorific performances of aqueous dispersing media are not as good as those of an oily medium (whether included in an emulsion or not). Indeed, generally the calorific capacity of oil is much lower than that of water and its thermal conductivity is also lower. Thus, in order to improve the efficacy of the ferrofluid composition in magnetic hyperthermia treatment, it would be preferable to use an oily ferrofluid composition or at least a dispersion of magnetic particles in an oil phase of an emulsion.

Moreover, systemic injection of aqueous ferrofluid in a patient poses the problem of the quantity of magnetic particles present at the target, which must be sufficient to compensate for thermal losses in the living environment, which is an aqueous environment thermoregulated at 37° C. Improving the efficacy of such a treatment by systemic administration would require the injection of a large quantity of injected nanoparticles, well beyond the recommended doses (of around 0.8 mg Fe/kg for iron oxide contrast agents) associated with a sufficient accumulation at the pathological area provided by favorable pharmacokinetics and effective targeting.

Ferrofluid nanoemulsions or ferrofluid oily suspensions are mainly known for use as MRI contrast agents and for cancer treatment by magnetic hyperthermia.

An example of ferrofluid in emulsion used as contrast agent is reported in application FR 3 001 154, which describes an oil-in-water nanoemulsion comprising an aqueous phase, a lipid phase comprising an oil, C6-C18 saturated fatty acid glycerides and magnetic particles based on an iron compound covered by one or more C8-C22 fatty acids and surfactants comprising at least one amphiphilic lipid and at least one targeting ligand. Such a nanoemulsion contains additives, such as surfactants, necessary for the stabilization of the nanoemulsion, that can alter the biocompatibility of the ferrofluid composition. Yet excellent biocompatibility is essential for medicinal use.

In this context, the Applicant sought to improve the biocompatibility of ferrofluid compositions for medicinal use and their efficacy (especially heat transfer by reduction of the dissipation rate of heat in the living environment in the context of use in magnetic induced hyperthermia) for the intended use, while having a chemical and colloidal stability at a temperature making it possible to restore the magnetic energy of the nanoparticles into thermal energy, and preferably compatible with a mode of administration by injection. The ferrofluid compositions of the invention can be envisaged for different medicinal applications and especially for cancer treatment by magnetic hyperthermia or as a contrast agent.

In order to improve the efficacy of ferrofluids, the Applicant is particularly interested in oily ferrofluids, especially in view of their application for magnetic hyperthermia or as a contrast agent, as well as ferrofluid emulsions.

Since iron oxide nanoparticles are biocompatible, the Applicant sought to improve the biocompatibility of the medium in which these nanoparticles are dispersed. In particular, the Applicant sought to use only biocompatible additives and liquid carrier and to avoid anything that is not biocompatible and could therefore lead to toxicity for the patient.

In this context, the invention proposes a novel biocompatible oily ferrofluid comprising iron-oxide based magnetic nanoparticles and an oil phase comprising at least one fatty acid ester, wherein said nanoparticles are surface functionalized by molecules of one or more phospholipids. Advantageously, in the oily ferrofluids according to the invention, iron-oxide based magnetic nanoparticles are dispersed and, preferably, in the form of a colloidal dispersion, in an oil phase containing at least one fatty acid ester.

Indeed, the formation of magnetic aggregates can lead to a reduction in heating efficiency of the nanoparticles under induction (i.e., under the effect of an alternating magnetic field). Thus, particular attention should be paid to the dispersion of these nanoparticles in order to prevent, as much as possible, the formation of aggregates, and especially large aggregates.

Thus, colloidal stability is a necessary condition to improve the performances of ferrofluid compositions, whether for their use in magnetic hyperthermia, as a contrast agent, or any other therapeutic application. In general, iron oxide nanoparticles are stabilized kinetically from the colloidal viewpoint under oily conditions and in emulsions using surfactants or dispersion agents, which prevents the formation of magnetic nanoparticle aggregates that can be detrimental to heating efficiency, especially due to magnetic dipolar interactions. Under the temperature conditions of use, magnetic nanoparticles should not flocculate under the effect of an applied alternating or static magnetic field and remain single phase. However, surfactants or dispersion agents are often poorly or not at all biocompatible.

The Applicant therefore sought the conditions for nanoparticle dispersibility at different temperatures for application in hyperthermia without using either surfactants or dispersion agents (or any other non-biocompatible additive). The Applicant thus worked on the surface chemistry of the metal oxide nanoparticles used. Indeed, the Applicant observed that a judiciously chosen chemical surface functionalization, combined with the presence of certain components in the oil phase, make it possible to significantly improve the colloidal stabilization of oily ferrofluid compositions under temperature conditions compatible with an injection.

Thus, the invention preferably concerns a biocompatible oily ferrofluid comprising iron-oxide based magnetic nanoparticles and an oil phase comprising at least one fatty acid ester, said iron-oxide based magnetic nanoparticles forming a colloidal dispersion in said oil phase from a temperature belonging to the range from 20 to 80° C., characterized in that said magnetic nanoparticles are surface functionalized by molecules of one or more phospholipids which do not completely cover the surface of the iron-oxide based magnetic nanoparticles and, in particular, which ensure a coverage rate of the surface of the iron-oxide based magnetic nanoparticles such that the fatty acid ester(s) present in the oil phase have access to the surface of the iron-oxide based magnetic nanoparticles.

The biocompatible oily fluid according to the invention advantageously has one or another of the following characteristics, alone or in combination, or even all the following characteristics:
the oily ferrofluid is free of water and/or free of surfactant;
the phospholipid molecules ensure a coverage of 19 to 76%, preferably of 29 to 76% and preferentially of 34 to 50%, of the surface of the iron-oxide based magnetic nanoparticles;
the surface density of functionalization (also called surface density of grafting) in phospholipid molecules belongs to the range from 0.32 molecule/nm² to 1.22 molecules/nm², preferably from 0.48 molecule/nm² to 1.22 molecules/nm²; and preferentially from 0.56 molecule/nm² to 0.79 molecule/nm²;
the phospholipid(s) contain at least one fatty chain, preferably 2 fatty chains, in particular C6-C30 and preferably C8-C24 or even C10-C22, and in particular C18 saturated or mono- or polyunsaturated, branched or, preferably, linear hydrocarbon chains;
the oil phase comprises at least 70% by mass of fatty acid ester(s), preferably the oil phase comprises from 80% to 95% by mass of fatty acid ester(s), relative to the total mass of the oil phase;
the fatty acid ester(s) of the oil phase are chosen from C6-C12, preferably C6-C10, saturated fatty acid triglycerides and C6-C12, preferably C6-C10 saturated fatty acid propylene glycols used singly or in mixture;
the content of magnetic nanoparticles belongs to the range from 0.01% to 50% by mass, preferably from 0.1% to 10% by mass, relative to the total mass of the oily ferrofluid; when it is a question of magnetic nanoparticle content, this only comprises iron oxide-based magnetic nanoparticles and does not include functionalization;
the magnetic nanoparticles are in the form of a spheroid, a polyhedron such as nanocubes, bipyramids or nanostars, a wafer, a nanorod, a nanodisk, or a nanoflower;
the phospholipid(s) have an —O(O)P(OH)O⁻ polar head and are preferably chosen from salts of 1,2-dioleoyl-sn-glycero-3-phosphatidic acid and 1,2-distearoyl-sn-glycero-3-phosphatidic acid;
the biocompatible oily ferrofluid further comprises a lipophilic active ingredient, in particular chosen from cancer treatment drugs, such as paclitaxel, docetaxel, or carmustine.

The functionalized iron-oxide based magnetic nanoparticles such as defined in the context of the invention, regardless of its variant of embodiment, are also an integral part of the invention.

The invention also concerns a process for preparing a biocompatible oily ferrofluid according to the invention.

The process for preparing a biocompatible oily ferrofluid according to the invention makes it possible to disperse the magnetic nanoparticles without using an additive that could be detrimental to the biocompatibility of the oily ferrofluid. Thus, advantageously, the process excludes any use of compounds that could induce toxicity for the patient other than the medicinal activity related to the presence of an agent for cancer treatment or induction treatment seeking to destroy cancer cells. Preferably, the fluids and solvents used are biocompatible and no non-biocompatible additive is used. Advantageously, no surfactant or dispersion agent is used in the process for preparing an oily ferrofluid according to the invention.

In order to obtain an oily ferrofluid that does not comprise surfactant or dispersion agent and which comprises magnetic nanoparticles in the form of a colloidal suspension at a temperature compatible with an injection, the inventors developed a particular process comprising the formation of a solvation layer of magnetic nanoparticles.

The Applicant has developed a process for preparing a biocompatible oily ferrofluid making it possible to optimize and control the degree of functionalization of the magnetic nanoparticles by the phospholipid molecules. Indeed, the Applicant has observed during their research that the choice of phospholipid molecules and the degree of functionalization had an impact on the stability and efficiency of the biocompatible oily ferrofluid.

Thus, the process for preparing a biocompatible oily ferrofluid according to the invention comprises the following successive steps:
a—providing an aqueous dispersion of iron-oxide based magnetic nanoparticles in an aqueous solvent which can be water or a mixture of water/solvent(s) miscible with water,
b—eliminating the aqueous solvent from the aqueous dispersion of magnetic nanoparticles,
c—obtaining a colloidal sol of magnetic nanoparticles by addition of solvent or a mixture of volatile organic solvents S2,
d—surface functionalizing said magnetic nanoparticles of the colloidal sol with molecules of at least one phospholipid,
e—eliminating said volatile organic solvent(s) S2 and dispersing the functionalized magnetic nanoparticles in an oil phase comprising at least one fatty acid ester.

The process for preparing a biocompatible ferrofluid according to the invention advantageously further comprises a step c2, after step c and before step d, of adding acid. This step allows increasing the affinity of the polar head of the phospholipid molecules with the surface of the magnetic nanoparticles in view of more efficient functionalization.

The invention also concerns a medicament and, in particular, a medicament for cancer treatment comprising a biocompatible oily ferrofluid according to the invention, or obtained by the process for preparing a biocompatible oily ferrofluid according to the invention.

The invention also concerns a biocompatible oily ferrofluid according to the invention or obtained according to the process for preparing a biocompatible oily ferrofluid according to the invention for its use during cancer treatment by magnetic induced hyperthermia. The invention also concerns a therapeutic method for cancer treatment by magnetic hyperthermia comprising the intratumoral injection of a biocompatible oily ferrofluid according to the invention, or a biocompatible oily ferrofluid obtained according to the preparation process according to the invention, then application of an external alternating magnetic field.

The invention also concerns an oil-in-water nanoemulsion comprising from 10% to 30% by mass of a biocompatible oily ferrofluid according to the invention, or a biocompatible oily ferrofluid obtained according to the preparation process according to the invention, an aqueous phase and at least one surfactant. Such a nanoemulsion can also comprise dispersion agents and/or targeting ligands. Preferably, the nanoemulsion is biocompatible.

The invention also concerns the preparation of a nanoemulsion according to the invention. This process comprises the following successive steps:

i—providing a biocompatible oily ferrofluid according to the invention or obtained according to the process for preparing a biocompatible oily ferrofluid according to the invention,
ii—providing an aqueous phase comprising at least one surfactant and
iii—mixing the aqueous phase and the biocompatible oily ferrofluid to form a nanoemulsion.

The nanoemulsion according to the invention or the nanoemulsion obtained according to the process according to the invention can be used in systemic administration for cancer treatment. Thus, the invention also concerns a medicament and, in particular, a medicament for cancer treatment comprising a nanoemulsion according to the invention, or obtained according to the process for preparing the nanoemulsion according to the invention. The invention also concerns a nanoemulsion according to the invention or obtained according to the process for preparing a nanoemulsion according to the invention for its use during cancer treatment by magnetic hyperthermia. The invention also concerns a therapeutic method for cancer treatment by magnetic hyperthermia comprising the systemic injection of a nanoemulsion according to the invention, or nanoemulsion obtained according to the preparation process according to the invention then application of a magnetic field.

Finally, the invention concerns a contrast product, in particular for magnetic resonance imaging (MRI) comprising a biocompatible oily ferrofluid according to the invention, or a nanoemulsion comprising the oily ferrofluid according to the invention.

Various other characteristics will appear from the description made below in reference to the attached drawings which show, by way of non-limiting examples, forms of embodiment of the subject of the invention.

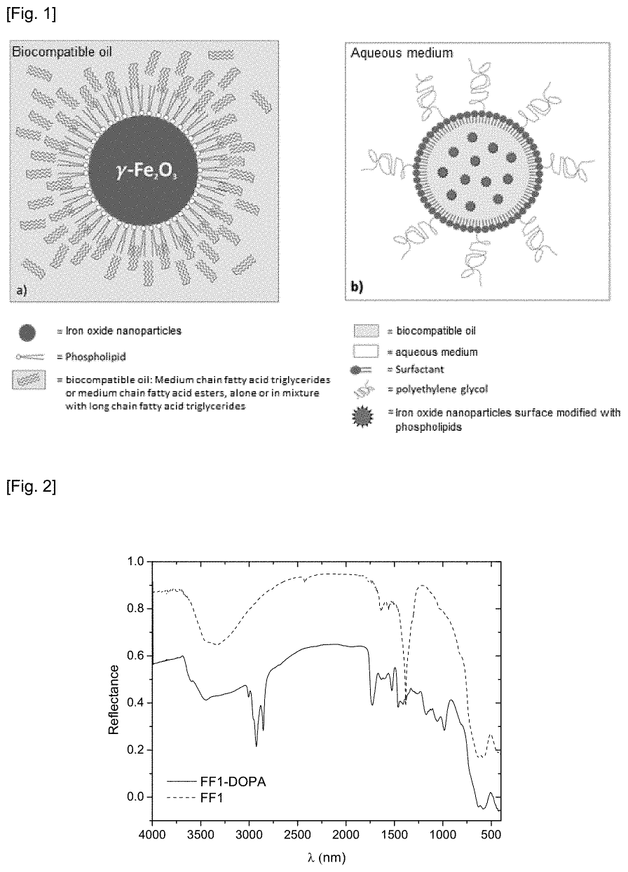

FIG. 1 is a schematic representation 1) of a ferrofluid according to the invention and 2) of a nanoemulsion according to the invention.

FIG. 2 represents diffuse reflection infrared spectroscopy (DRIFT) analyses of non-functionalized magnetic nanoparticles and magnetic nanoparticles functionalized with DOPA.

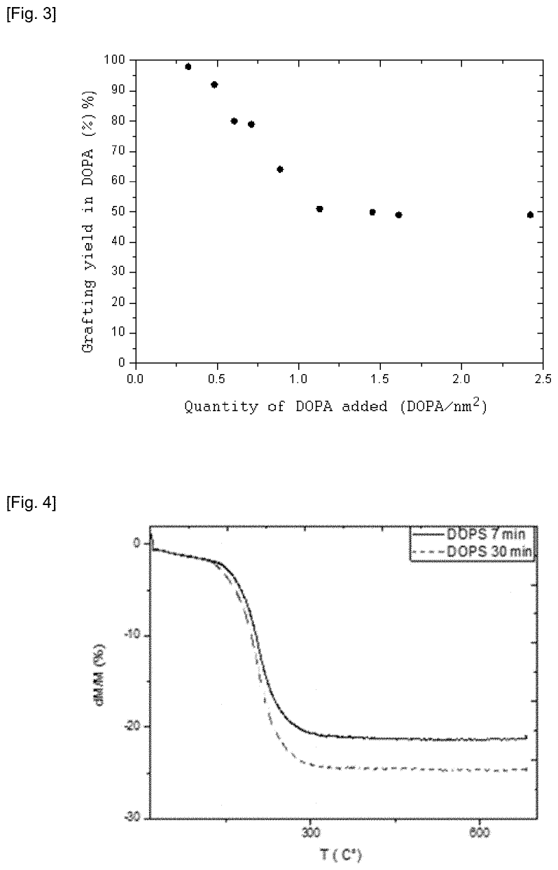

FIG. 3 represents the chemisorption yields in DOPA determined by TGA measurements for different quantities of spheroid nanoparticles FF1 involved in the presence of acid.

FIG. 4 represents the thermograms of functionalized nanoparticles corresponding to 2000% of the quantity necessary to form a monolayer of DOPS at 7 min and 30 min of reaction.

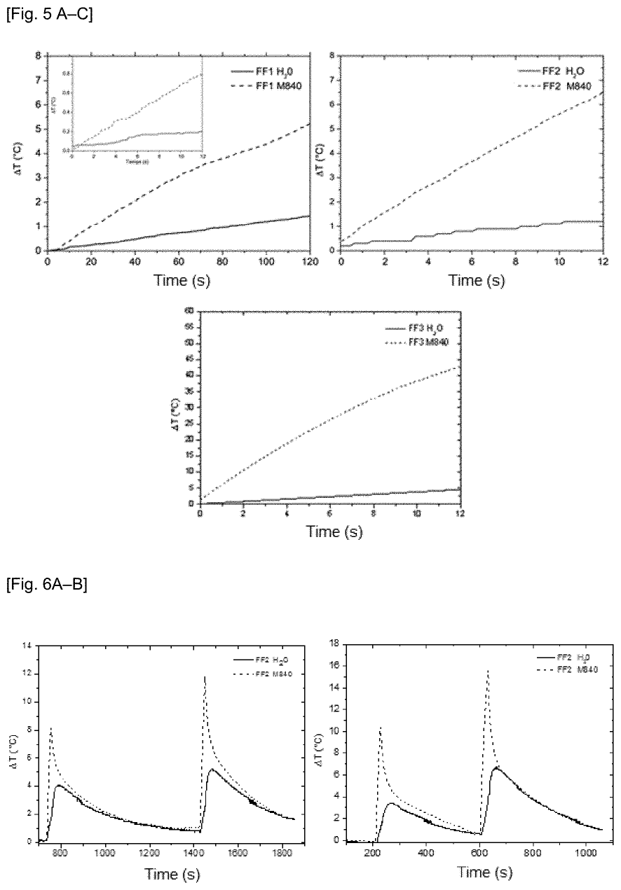

FIGS. 5A, 5B and 5C represent the kinetic temperature profiles of dispersions of spheroid nanoparticles FF1 and dispersions of nanoflower morphology FF2 and FF3, respectively, subjected to magnetic induction (of field-frequency pair of: (FIG. 5A) 755 kHz, 10.2 kA/m, (FIG. 5B) 473.5 kHz, 13.36 kA/m and (FIG. 5C) 473.5 kHz, 13.36 kA/m) dispersed in water or in Miglyol M840® after functionalization by DOPA with theoretical phospholipid molecule coverage densities of, respectively, 0.81 and 1.29 and 1.10 molecules/nm² (regulation temperature T₀=37° C. and CFe₂O₃=5 g/L where CFe₂O₃ is the mass concentration in Fe₂O₃). In the insert of FIG. 5A, enlargement of the temperature profile over the range [0-10 s].

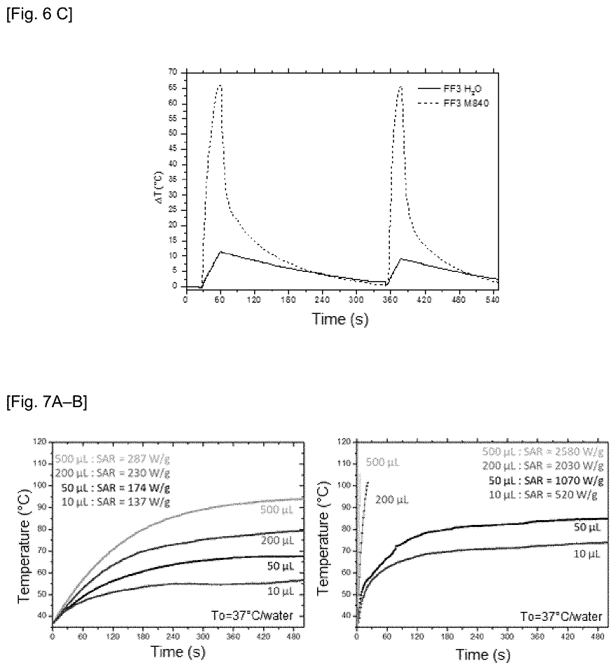

FIGS. 6A, 6B and 6C represent the kinetic temperature profiles of dispersions of nanoparticles of nanoflower morphology FF2 and FF3 dispersed in water or in Miglyol M840® after functionalization by DOPA with a nominal surface coverage density in phospholipid molecules, respectively, of 1.29 and 1.10 molecules nm² subjected for each of the dispersions to two cycles of magnetic induction (field/frequency pair of: (FIG. 6A) nanoparticle dispersion FF2 at 473.5 kHz, 13.36 kA/m, (FIG. 6B) nanoparticle dispersion FF2 at 344.5 kHz, 16.23 kA/m and (FIG. 6C) nanoparticle dispersion FF3 at 473.5 kHz, 13.36 kA/m, regulation temperature of T₀=37° C. and CFe₂O₃=5 g/L).

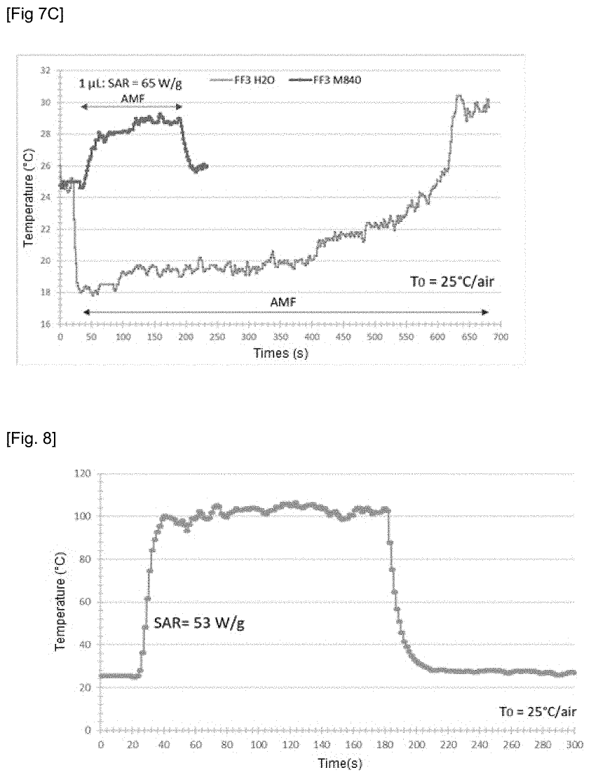

FIGS. 7A and 7B represent the kinetic temperature profiles and the values of thermal power dissipated by the nanoparticles (represented by the specific absorption rate (SAR)) of the nanoparticle dispersions of nanoflower morphology FF3 according to the volume of the dispersion medium: (FIG. 7A) in water or (FIG. 7B) in Miglyol M840® after functionalization by DOPA for a nominal surface coverage density in phospholipid molecules of 1.10 molecules/nm², subjected for each of the dispersions to a magnetic induction of field/frequency pair of 473.5 kHz, 13.36 kA/m, a regulation temperature of T₀=37° C. and a mass concentration of iron oxide CFe₂O₃=5 g/L.

FIG. 7C represents the kinetic profiles corresponding to 1 μL of the nanoparticle dispersion FF3 at 5 g/L in water and in Miglyol M840®, deposited directly at the tip of the magnetic induction temperature sensor (called MFA: 473.5 kHz; 13.36 kA/m, T₀=25° C., ambient room temperature).

FIG. 8 represents the temperature kinetic profile of the nanoparticles FF3 dispersed in Miglyol M840®, after functionalization by DOPA for a nominal surface coverage density in phospholipid molecules of 1.10 molecules/nm², of mass concentration of iron oxide Fe₂O₃ of 300 g/L and for a volume of 1 μL deposited at the tip of the temperature sensor (T₀=25° C., ambient room temperature).

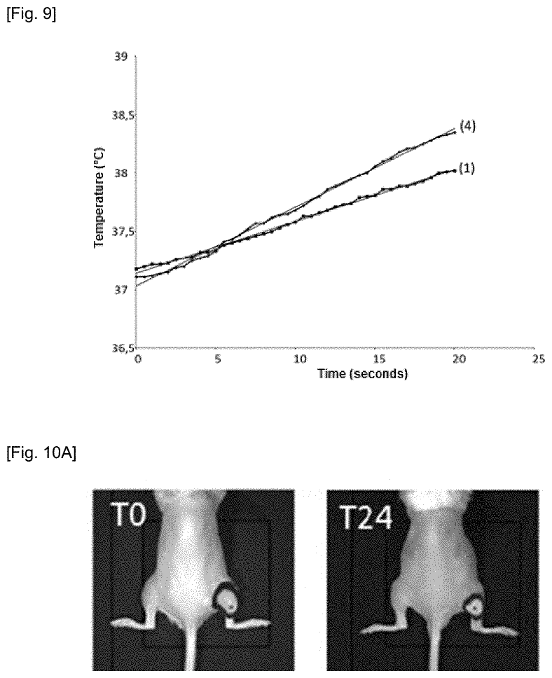

FIG. 9 represents the kinetic temperature profiles of the nanoemulsions (1) and (4) (CFe₂O₃=12 g/L) containing the spheroid nanoparticles FF1 dispersed in Miglyol M840 emulsified in aqueous phase, under induction at 473.5 KHz, 13.36 kA/m (regulation temperature T₀=37° C.) for 20 seconds.

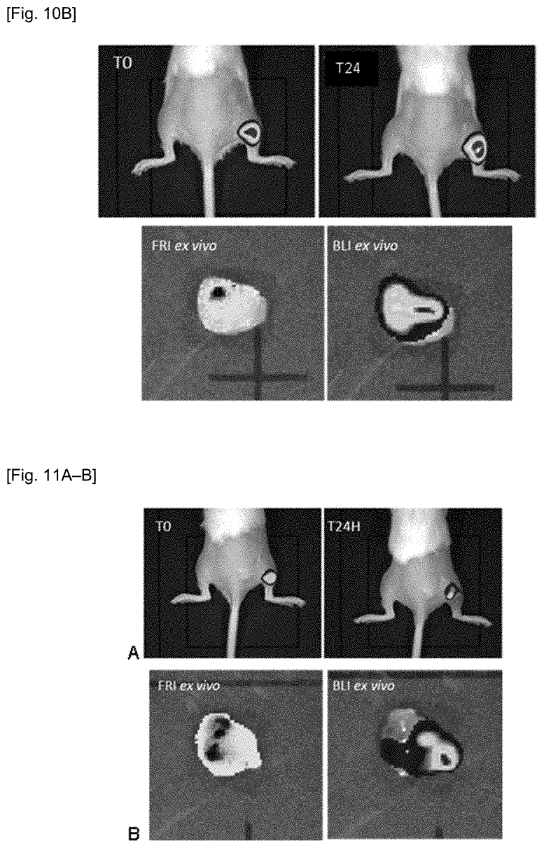

FIG. 10A shows the bioluminescence images produced following the intratumoral injection of the oily ferrofluid as described in Example 6, in a mouse subcutaneous tumour (two injections of 1 μL of oily ferrofluid at 300 g/L at the same injection site before (TO) and 24 h after (T24) induction treatment (473.5 KHz, 13.36 kA/m). FIG. 10B shows the fluorescence reflectance imaging (FRI) and bioluminescence imaging (BLI) images of the tumour ex vivo more than 24 h after induction treatment

FIG. 11A shows the bioluminescence images produced following the intratumoral injection of the oily ferrofluid as described in Example 6, in a mouse subcutaneous tumour (three injections of 1 μL of oily ferrofluid at 300 g/L at different places on the tumour) before (TO) and 24 h after (T24) induction treatment (473.5 KHz, 13.36 kA/m). FIG. 11B shows the fluorescence (FRI) and bioluminescence (BLI) images of the tumour ex vivo more than 24 h after induction treatment

BIOCOMPATIBLE OILY FERROFLUID

The invention concerns a biocompatible oily ferrofluid comprising functionalized magnetic nanoparticles in suspension in an oil phase comprising at least one fatty acid ester, such as diagramed in FIG. 1.

The functionalized magnetic nanoparticles used in the context of the invention are iron-oxide based nanoparticles which are surface functionalized by molecules of at least one phospholipid. Iron oxide has the advantage of being biocompatible. Advantageously, the nanoparticles used in the context of the invention do not comprise any metal element that could be toxic, such as cobalt or manganese.

In the context of the invention, “nanoparticles” refers to particles of nanometric elementary size, i.e., with an average elementary size greater than 1 nm and less than 100 nm preferably exhibiting a monomodal size distribution with a standard deviation of less than 30% in number relative to the mean value. Elementary size means the largest dimension of the nanoparticle. In the context of the invention, the elementary size of a functionalized magnetic particle corresponds to the dimension of the iron-oxide based nanoparticle as such and does not comprise the functionalization with phospholipid(s) molecules.

Preferably, in the context of the invention, the magnetic nanoparticles have an average elementary size less than 30 nm, preferably less than 25 nm. Preferably, the magnetic nanoparticles used in the context of the invention have an average elementary size greater than 3 nm, preferably greater than 5 nm. Preferably, the functionalized magnetic nanoparticles have an average elementary size belonging to the range from 5 to 34 nm, preferably from 7 to 24 nm.

“Average elementary size” means the average size of the inorganic nanoparticles without the phospholipid coating and not aggregated. The average elementary size corresponds to the arithmetic mean of the elementary sizes measured in a set of nanoparticles, in particular in a set of 200 nanoparticles. The elementary size of nanoparticles can be measured by transmission electron microscopy (TEM), after elimination of the oil phase.

In the oily ferrofluids according to the invention, the magnetic nanoparticles based on functionalized iron oxide in accordance with the invention form a colloidal dispersion in the oil phase used, from a temperature belonging to the range from 20 to 80° C., preferably from a temperature equal to 60 or 70° C., preferentially from a temperature equal to 37° C., and still more preferably, from a temperature equal to 20 or 25° C. Conventionally, colloidal dispersion means solid particles dispersed in a liquid phase that is stable for at least 24 hours, i.e., which does not sediment or aggregate. The colloidal stability of magnetic ferrofluid dispersions as a function of temperature of the medium can be characterized qualitatively by their clarity and translucence, as opposed to a suspension of aggregates or flocculates that has a turbid, cloudy or opaque character. A quantitative evaluation can be done by measuring transmittance at 800 nm. For example, nanoparticle dispersions of mass concentration of 0.2 g/L in Fe₂O₃—γ in the size range from 5 to 15 nm is qualified unstable when such dispersions have lost at least 30% of their transmittance at 800 nm relative to that of a stable dispersion of the same concentration. Such transmittance measurements can be performed with a VARIAN Cary 500 spectrophotometer equipped with a temperature regulator.

The colloidal stability as a function of the surface coverage rate in phospholipid molecules can also be qualified by dynamic light scattering (DLS) measurement that makes it possible to obtain the hydrodynamic size of nanoparticles by considering the nanoparticle solvation sphere. Thus, for nanoparticles of 10 nm, hydrodynamic diameters of 40 nm to 50 nm can be measured for optimal conditions, around 100 nm for conditions at the limit of the domain of stability and much higher than 100 nm when the dispersions are not stable.

Other techniques are used to obtain information on the nanoparticles such as X-ray diffraction (XRD) which makes it possible to obtain the size of the crystal coherence domain according to the protocols described in the examples.

The nanoparticles used in the context of the invention can be of various shapes, such as in the shape of a spheroid, a polyhedron such as nanocubes, bipyramids or nanostars, a wafer, a nanorod, a nanodisk or a nanoflower. So-called nanoflower nanoparticles have a typical morphology in the form of a flower resulting from the assembly of epitaxial nanocrystallites forming multiple buddings.

In the context of the invention, “spheroid” means spherical or quasi-spherical, i.e., which has an index of sphericity (i.e., the ratio between its largest diameter and its smallest diameter) less than 1.2.

According to a first preferred embodiment of the invention, the functionalized magnetic nanoparticles are in spheroid form, and in particular spherical or quasi-spherical. According to this embodiment, the magnetic nanoparticles then have an average elementary size belonging to the range from 5 to 20 nm, preferably from 7 to 15 nm.

According to a second preferred embodiment of the invention, the functionalized nanoparticles used in the context of the invention are in the nanoflower form, preferably monocrystalline or quasi-monocrystalline. According to this embodiment, the magnetic nanoparticles then have an average elementary size belonging to the range from 10 to 34 nm, preferably from 10 to 24 nm.

In the context of the invention, the nanoparticles used are iron-oxide based nanoparticles surface functionalized by molecules of at least one phospholipid. According to a particular embodiment, the magnetic nanoparticles are nanoparticles composed solely of iron oxide whose surface is functionalized by phospholipid molecules.

In the context of the invention, “iron-oxide based” nanoparticles means nanoparticles essentially made up of iron oxide, or even exclusively made up of iron oxide.

The iron oxide particles can be ferrimagnetic nanoparticles, and typically magnetite particles (Fe₃O₄) or maghemite particles (γ—Fe₂O₃) or any other cubic sesquioxide of formula [Fe³⁺]_{Td}[Fe³⁺_{1+2 z/3}Fe²⁺_{1-z}V_{z/3}]_{Oh}O₄ (Td and Oh representing, respectively, the tetrahedral and octahedral sites of the spinel and V the cation vacancies) in which z varies from 0 to 1. Preferably, the iron oxide nanoparticles are nanoparticles of maghemite, i.e. a cubic sesquioxide of formula

[Fe³⁺]_{Td}[Fe³⁺_{1+2 z/3}Fe²⁺_{1-z}V_{z/3}]_{Oh}O₄with z=1.

In the context of the invention, the magnetic nanoparticles of the biocompatible oily ferrofluid are surface functionalized by one or more phospholipids, i.e., identical or different phospholipid molecules are linked by chemical bond to the surface of each magnetic nanoparticle. These chemical bonds are preferably coordination or complexation bonds established with the polar head of phospholipid molecules and a surface iron site. In particular, the phospholipid head, especially of —O(O)P(OH)O⁻ type is bound by one or more coordination bonds to the iron-oxide based nanoparticles.

“Phospholipid” conventionally means a molecule made up of a phosphate type polar head and one or more fatty chains. Preferably, the phospholipid molecules are bound via their polar head to the surface of the nanoparticle.

Preferably, the magnetic nanoparticles of the biocompatible oily ferrofluid are surface functionalized by a single type of phospholipid. Although not preferred, functionalizing the magnetic nanoparticles by two or more different nanoparticles can be envisaged.

In the context of the invention, the phospholipid molecules contain at least one fatty chain, preferably at least two fatty chains and, in particular, two fatty chains. These fatty chains are typically saturated or unsaturated, linear or branched hydrocarbon chains comprising 6 to 30 carbon atoms. These fatty chains can be different or, preferably identical for each phospholipid used.

Preferably, the fatty chains of the phospholipid molecules are linear. Without wishing to be bound by any theory, the Applicant is of the opinion that these fatty chains of phospholipid molecules are intercalated with the fatty chains of fatty acid esters of the oil phase, thus allowing the creation of a solvation sphere. The absence of branching on the fatty chains of the phospholipid molecules facilitates this intercalation.

The fatty chains of the phospholipid molecules can be saturated or mono or polyunsaturated. Advantageously, the fatty chains of the phospholipid molecules are monounsaturated.

The fatty chains of the phospholipid molecules advantageously comprise from 6 to 30 carbon atoms, preferably 8 to 24 carbon atoms, preferably 10 to 22 carbon atoms and, in particular, 18 carbon atoms.

In the context of the invention, the functionalization of the magnetic particles preferably occurs via the polar head of the phospholipid molecules: a chemical bond between the iron oxide sites present on the surface of the magnetic nanoparticles and the polar head of the phospholipid molecules allows a strong anchoring and a solid functionalization. Thus, this surface functionalization confers a good chemical stability and is not deteriorated under physiological and storage conditions.

The polar head of the phospholipid molecules used to surface functionalize the magnetic particles of the oily ferrofluid according to the invention are phosphate fragments and preferably have a low steric hindrance. The Applicant has actually observed during his research that a low steric hindrance allows better control of the kinetics and the functionalization rate. Examples of the polar head of the phospholipid molecules which can be bound to the surface of the magnetic nanoparticles according to the invention include the phosphate fragments derived from phosphoric acid, phosphatidylethanolamine, phosphatidylethanol, phosphothioethanol or salts thereof. In a particularly preferred manner, the polar head is the —O(O)P(OH)O⁻ group.

The phospholipid molecules can advantageously be chosen from the salt forms of glycerophospholipids, such as for example 1,2-dioleoyl-sn-glycero-3-phosphatidic acid (DOPA), 1,2-dioleoyl-sn-glycero-3-phosphatidyl-L-serine, 1,2-dioleoyl-sn-glycero-3-phosphatidylethanolamine, 1,2-distearoyl-sn-glycero-3-phosphatidic acid (DSPA), phosphatidylinositol, 1,2-dipalmitoyl-sn-glycero-3-phosphothioethanol, 1,2-dioleoyl-sn-glycero-3-phosphoethanolamine-N-[3-(2-pyridyldithio)propionate], or derived from sphingolipids such as sphingomyelin. DOPA and DSPA in the anionic form are particularly preferred.

Preferably, the phospholipid molecules ensure a coverage of from 19 to 76%, preferably 29 to 76%, and preferentially 34 to 50%, of the surface of iron-oxide based magnetic nanoparticles.

The surface density of functionalization of the magnetic nanoparticles by phospholipid molecules preferably belongs to the range from 0.32 molecule/nm² to 1.22 molecules/nm², preferably 0.48 molecule/nm² to 1.22 molecules/nm², and preferentially 0.56 molecule/nm² to 0.79 molecule/nm². The functionalization density can be determined as in the examples, using a thermogravimetric analysis (TGA).

According to one embodiment, the nanoparticles are of spheroid morphology and the surface density of functionalization of these nanoparticles by phospholipid molecules preferably belongs to the range from 0.32 molecule/nm² to 1.22 molecules/nm², preferably 0.48 molecule/nm² to 1.22 molecules/nm², and preferentially 0.56 molecule/nm² to 0.79 molecule/nm².

According to a particularly preferred embodiment, the ferrofluids according to the invention have the following characteristics:
the magnetic nanoparticles are of spheroid morphology, preferably with an average elementary size belonging to the range from 5 to 20 nm, preferably from 7 to 15 nm,
the polar head of the phospholipid molecules is the —O(O)P(OH)O⁻ group; and, preferably, the nanoparticles are functionalized with the phosphate anion of 1,2-dioleoyl-sn-glycero-3-phosphatidic acid, or 1,2-distearoyl-sn-glycero-3-phosphatidic acid,
the surface density of coverage of the magnetic nanoparticles by phospholipid molecules belongs to the range from 0.32 molecule/nm² to 1.22 molecules/nm², preferably from 0.48 molecule/nm² to 1.22 molecules/nm², and preferentially from 0.56 molecule/nm² to 0.79 molecule/nm²,
the fatty acid ester is chosen from triglycerides of caproic, capric and caprylic acids and propylene glycol esters of caproic, capric and caprylic acid, and mixtures thereof,
the oil phase comprises at least 70% by mass of fatty acid ester(s), preferably the oil phase comprises from 80% to 95% by mass of fatty acid ester(s), relative to the total mass of the oil phase,
the fatty acid ester(s) of the oil phase are chosen from C6-C12, preferably C6-C10 saturated fatty acid triglycerides and C6-C12, preferably C6-C10 saturated fatty acid propylene glycols, used alone or as a mixture.

According to another embodiment, the nanoparticles are of nanoflower morphology.

According to a first embodiment, the functionalized magnetic nanoparticles present in the biocompatible oily ferrofluid according to the invention are maghemite nanoparticles surface functionalized by phospholipid molecules having a phosphatidylserine polar head and at least one, preferably two C10-C22, especially C18, unsaturated and, preferably, monounsaturated linear fatty chains. Preferably, according to this embodiment, the maghemite nanoparticles are surface functionalized by 1,2-dioleoyl-sn-glycero-3-phosphatidyl-L-serine. According to this embodiment, the effective surface density of phospholipid molecule coverage on each nanoparticle belongs, for example, to the range of from 0.48 to 1.22 molecules/nm² and preferably from 0.56 to 0.79 molecules/nm².

According to a preferred second embodiment, the functionalized magnetic nanoparticles present in the biocompatible oily ferrofluid according to the invention are maghemite nanoparticles surface functionalized by phospholipid molecules having a —O(O)P(OH)O polar head and at least one, preferably two, C10-C22, especially C18, unsaturated and, preferably, monounsaturated linear fatty chains. Preferably, according to this embodiment, the maghemite nanoparticles are surface functionalized by the salt form of 1,2-dioleoyl-sn-glycero-3-phosphatidic acid. According to this embodiment, the surface density of coverage by phospholipid molecules on each nanoparticle advantageously belongs to the range from 0.32 molecule/nm² to 1.22 molecules/nm², preferably 0.48 molecule/nm² to 1.22 molecules/nm², and preferentially 0.56 molecule/nm² to 0.79 molecule/nm².

Preferably, the magnetic nanoparticle content (this only includes iron oxide-based magnetic nanoparticles and does not include functionalization) in the oily ferrofluid belongs to the range from 0.01 to 50% by mass relative to the total mass of oily ferrofluid, preferably from 0.1 to 10% by mass. In other words, the mass content of magnetic nanoparticles per litre of biocompatible oily ferrofluid advantageously belongs to the range from 0.01 g/L to 500 g/L, preferably from 1 g/L to 100 g/L.

In the context of the invention, the functionalized nanoparticles are dispersed or suspended in an oil phase, and form a colloidal dispersion. This oil phase is advantageously water-free, i.e., it comprises less than 0.1% by mass of water, preferably less than 0.05% by mass of water, and better still less than 0.01% by mass of water relative to the total mass of oil phase.

The oil phase comprises at least one fatty acid ester and, preferably, at least 70% by mass of fatty acid esters, preferably 80% to 95% by mass of fatty acid esters, relative to the total mass of the oil phase.

In the context of the invention, the fatty acid esters are esters of carboxylic acid with aliphatic chain comprising 4 to 36 carbon atoms.

The fatty chains of the fatty acid esters can be either mono- or polyunsaturated, or saturated. A mixture of saturated and unsaturated fatty acid esters can also be used in the context of the invention.

The fatty chains of fatty acid esters can be branched or, preferably, linear. Without wishing to be bound by any theory, the Applicant is of the opinion that these fatty chains of the fatty acid esters are intercalated with the fatty chains of the phospholipid molecules of functionalized nanoparticles, thus allowing the creation of a solvation sphere. The absence of branching on the fatty chains of the fatty acid esters facilitates this intercalation.

Preferably, in the context of the invention, the fatty acid esters are chosen from C6-C18 saturated fatty acid esters, preferably C6-C12 and, in particular, C6-C10. Preferably, the fatty acid esters are chosen from C6-C12 saturated fatty acid esters, and, in particular, C6-C10. By way of examples, the fatty acid of the fatty acid ester can be chosen from caproic acid, caprylic acid, capric acid, lauric acid, palmitic acid, stearic acid, oleic acid, linoleic acid, linolenic acid and docosahexaenoic acid.

Preferably, in the context of the invention, the fatty acid esters are chosen from C6-C18 saturated fatty acid triglycerides, preferably C6-C12 and, in particular, C6-C10 and C6-C12 saturated fatty acid propylene glycols, and in particular C6-C10, used alone or as a mixture. Preferably, the fatty acid esters are chosen from C6-C12 saturated triglycerides, and, in particular, C6-C10 and C6-C12 saturated fatty acid propylene glycols, and in particular C6-C10, used alone or as a mixture.

Examples of fatty acid esters that can be used in the context of the invention include triglycerides of saturated fatty acids such as triglycerides of caproic, capric or caprylic acids, or propylene glycols of saturated fatty acids such as propylene glycol dicaprylocaprate, used alone or as a mixture.

According to a first embodiment, the oil phase comprises a single fatty acid ester chosen from C6-C10 fatty acid triglycerides and C6-C10 fatty acid propylene glycols. According to this embodiment, the fatty acid ester is advantageously chosen from caproic, capric and caprylic acid triglycerides and the propylene glycol ester from caproic, capric and caprylic acid.

According to a second embodiment, the oil phase comprises a mixture of fatty acid esters, in particular 2 or 3 fatty acid esters. According to this embodiment, the fatty acid esters are advantageously chosen from those of caprylic acid, capric acid and lauric acid.

The oil phase can also comprise one or more oils different from said fatty acid ester, and optionally one or more lipophilic additives. Advantageously, these additional oils and lipophilic additives are biocompatible and do not generate any toxicity for the patient when the oily ferrofluid according to the invention is administered to them. According to a particular embodiment, the oily ferrofluid according to the invention does not comprise surfactant or dispersion agent.

Any biocompatible oil can be used in the context of the invention.

For example, the oil phase of the biocompatible oily ferrofluid can comprise an oil chosen from soybean oil, olive oil, sesame oil, cottonseed oil, poppyseed oil, copra oil, coconut oil, flaxseed oil, sunflower oil, C6-C12 saturated fatty acid triglyceride oils, and fish oils.

According to a preferred embodiment of the invention, the oil phase comprises at least one fatty acid ester and at least one oil, and preferably a single oil different from the fatty acid ester. According to this embodiment, the fatty acid ester is preferably chosen from triglycerides of capric or caprylic acids, propylene glycol dicaprylocaprate, used alone or as a mixture.

Preferably, the oil phase of the biocompatible oily ferrofluid according to the invention comprises less than 30% by mass of oils other than the at least one fatty acid ester relative to the total mass of the oil phase, and preferably 20 to 5% by mass.

The biocompatible oily ferrofluid can also comprise other biocompatible lipophilic additives. Examples of biocompatible lipophilic additives include lipophilic active ingredients, such as cancer treatment lipophilic active ingredients such as paclitaxel, docetaxel, or carmustine. Other biocompatible lipophilic additives, known in the state of the art, may be used in the context of the invention and will not be detailed here.

Preferably, the mass percentage of magnetic nanoparticles in the oil phase belongs to the range from 0.01% to 50%, preferably from 0.1% to 10% relative to the total mass of oily ferrofluid.

Advantageously, the biocompatible oily ferrofluid according to the invention is stable: the magnetic nanoparticles are well dispersed, do not flocculate from a temperature belonging to the range from 20 to 80° C. and in particular from 37° C. and better still from 25 or even 20° C. The colloidal dispersions are obtained under atmospheric pressure (1013.25 hPa). When we say that said functionalized iron— oxide based magnetic nanoparticles form a colloidal dispersion in said oil phase from a temperature belonging to the range from 20 to 80° C., this does not mean that the colloidal suspension is obtained over this entire temperature range. This means that stability is obtained at least at one temperature in the range, and in particular over at least part of the range, in particular in the high temperature range for which all the nanoparticles described disperse and release all their heat under magnetic induction. However, it is preferable that the functionalized iron— oxide based magnetic nanoparticles form a colloidal dispersion in said oil phase at ambient temperature, and especially when the temperature belongs to the range of 20-25° C. Advantageously, the stability of the colloidal dispersions is maintained for at least 24 hours, preferably for at least one month after being obtained. The colloidal character of the dispersion and its stability can be verified under ambient air and at atmospheric pressure (1013.25 hPa) especially over a duration of 24 hours.

In the context of the invention, the temperature range in which the biocompatible oily ferrofluid is stable is dependent on the surface density of functionalization by phospholipid molecules, their chemical nature and the size and morphology of the magnetic nanoparticles.

Process for Preparing a Biocompatible Oily Ferrofluid According to the Invention.

The invention also concerns the process for preparing a biocompatible oily ferrofluid according to the invention such as detailed above. This process comprises the following successive steps:
a—providing an aqueous dispersion of iron-oxide based magnetic nanoparticles in an aqueous solvent which can be water or a mixture of water/solvent(s) miscible with water,
b—eliminating the aqueous solvent from the aqueous dispersion of magnetic nanoparticles,
c—obtaining a colloidal sol of magnetic nanoparticles by addition of solvent or a mixture of volatile organic solvents S2,
d—surface functionalizing said magnetic nanoparticles of the colloidal sol with molecules of at least one phospholipid,
e—eliminating said volatile organic solvent(s) S2 and dispersing the functionalized magnetic nanoparticles in an oil phase comprising at least one fatty acid ester.

At step a, the magnetic nanoparticles are not functionalized by the phospholipid molecules such as described above for the functionalized nanoparticles of the biocompatible oily ferrofluid according to the invention. These nanoparticles, after a surface functionalization step (step d), correspond to functionalized nanoparticles of the oily ferrofluid according to the invention. These non-functionalized nanoparticles therefore comprise iron oxides, preferably are solely made up of iron oxides and, in particular, chosen from the iron oxides described for the functionalized magnetic nanoparticles of the biocompatible oily ferrofluid according to the invention. In aqueous medium, said iron oxides can be hydrolyzed on the surface of said nanoparticles generating iron hydroxyl sites on the surface. When they are dispersed in aqueous phase, these iron oxide nanoparticles then have at their surface positive or negative electrostatic charges depending on the pH values of the medium. Moreover, these non-functionalized nanoparticles have the same form as the functionalized nanoparticles of the biocompatible oily ferrofluid according to the invention.

These non-functionalized nanoparticles are in suspension in aqueous medium. The aqueous medium could notably be composed of water optionally in mixture with one or more solvents miscible with water such as methanol, ethanol, isopropanol, tetrahydrofuran, dimethylsulfoxide, dimethylformamide, acetonitrile, acetone or ethyl acetate. Preferably, the aqueous medium is composed solely of water and can optionally comprise spectator counterions resulting from the stabilization of the ferrofluid in an acid medium (such as nitrate or perchlorate and their acid form at pH 2.5) or in a basic medium (such as potassium, tetramethylammonium and their corresponding base at pH 10).

The magnetic nanoparticle concentration in the aqueous dispersion is not limiting. Advantageously it is greater than 1 g/L, preferably greater than 10 g/L, or even greater than 100 g/L.

Preferably, the aqueous dispersion of non-functionalized nanoparticles is solely made up of an aqueous solvent and non-functionalized nanoparticles. Although not preferred, the aqueous dispersion of non-functionalized nanoparticles can also comprise hydrophilic additives, such as steric stabilizing agents of macromolecular origin such as poly(vinylpyrrolidone), dextran, functionalized poly(ethylene oxide) or surfactants such as β-octyl glucoside, Tween® 80 or sodium dodecyl sulphate.

The aqueous dispersion of non-functionalized iron-oxide based magnetic nanoparticles can be prepared by any technique known to the skilled person.

Step b of the process for preparing the biocompatible oily ferrofluid according to the invention consists of eliminating the aqueous solvent from the aqueous dispersion of non-functionalized magnetic nanoparticles. In other words, this step consists of eliminating the water used as solvent (or any other solvent miscible with water optionally present in the aqueous medium) in the aqueous dispersion of non-functionalized nanoparticles, as well as physiosorbed water, thus making it possible to obtain aggregates of non-functionalized magnetic nanoparticles with no solvent.

For this, different techniques known in the state of the art may be used:
use of a magnet in order to separate the metallic nanoparticles from the aqueous medium, the medium being removed by suction or by pipetting for example,
extraction on a magnetic column,
ultrafiltration,
centrifugation,
successive washes with an organic solvent miscible with water such as methanol, ethanol, isopropanol, tetrahydrofuran, dimethylsulfoxide, dimethylformamide, acetonitrile, acetone, or ethyl acetate,
freeze-drying,
pervaporation.

Preferably, the water and/or solvents miscible with water optionally present are eliminated following flocculation of the nanoparticles. Flocculation is obtained when the medium is neutralized or by addition of a solvent miscible with water such as acetone or ethanol. The aqueous medium comprising water is then separated from the flocculated nanoparticles. For example, a magnet is used in order to retain the aggregates of non-functionalized magnetic nanoparticles, while the medium is suctioned or pumped using a membrane pump, for example.

Thus, regardless of the method for eliminating the aqueous solvent used, successive washings with a volatile polar organic solvent miscible with water (solvent called S1 below), such as ethanol, can enable all the physiosorbed water to be eliminated from the nanoparticles.

Advantageously, this water elimination step makes it possible to recover non-functionalized magnetic nanoparticles with a water content less than 0.25%, preferably less than 0.025% by mass relative to the mass of the non-functionalized magnetic nanoparticles according to their developed surface.

Step c of the preparation process consists of preparing a colloidal sol of non-functionalized magnetic nanoparticles.

In the context of the invention, “colloidal sol” means a homogeneous colloidal dispersion of solid particles in a continuous liquid medium.

The colloidal sol of non-functionalized nanoparticles can be created by dispersing the non-functionalized nanoparticles in a dispersive medium.

The dispersion can be performed by any technique known to the skilled person, such as vortexing and homogenization in an ultrasonic bath.

Preferably, the dispersive medium also allows solubilizing the phospholipid(s) used subsequently to surface functionalize the magnetic nanoparticles. Also, the dispersive medium comprises, or is even exclusively made up of, one or more volatile organic solvents (called solvents S2). Indeed, such volatile organic solvents allow both the dispersion of the non-functionalized nanoparticles as well as the solubilization of the phospholipids that will be used during functionalization step d: thus, the kinetics, yield of the surface functionalization reaction and the control of the phospholipid grafting rates are greatly improved.

Preferably, the dispersive medium used and, in particular, the organic volatile solvent(s) S2, is biocompatible. Thus, if traces of this dispersive medium are still present in the oily ferrofluid according to the invention, no toxicity for the patient is generated.

Preferably, the dispersive medium is volatile in order to facilitate its elimination by evaporation under reduced pressure during step e of the process. Thus, for the case where the dispersive medium is not biocompatible, no toxicity is due to this medium.

Examples of volatile organic solvent type dispersive medium S2 include chloroform alone or in mixture with methanol (in a ratio of 2:1 v/v, for example), diethyl ether, heptane, dichloromethane or isopropanol.

According to a particular embodiment, the dispersive medium is made up of a single volatile organic solvent S2, preferably polar. According to this embodiment, the dispersive medium is advantageously chloroform.

Preferably, the magnetic nanoparticle concentration in the colloidal sol belongs to the range from 0.1 to 50% by mass relative to the total mass of the colloidal sol, preferably from 1 to 10% by mass. Such magnetic nanoparticle concentrations advantageously allow a good production yield and control of the grafting rate in terms of the distribution of phospholipid molecules on the surface of the magnetic nanoparticles following the functionalization step.

An optional step c2, performed after step c and before step d can be performed, especially in order to promote the complexation of the polar phosphatide head of the phospholipid molecules with the iron sites present on the surface of the magnetic nanoparticles, in view of the nanoparticle functionalization step (step d). To do this, an organic acid is added to the non-functionalized colloidal sol of magnetic nanoparticles. Preferably, a weak organic acid is used. Weak organic acid means an acid whose dissociation reaction in water is not complete. The chemisorption kinetics of the phospholipid molecules are improved.

Advantageously, the organic acid used for this step is biocompatible.

Preferably, the organic acid used for this optional step c2 is chosen from weak organic acids miscible in the volatile organic solvent S2 used in step c to obtain the colloidal sol, and advantageously at less than 10% by volume.

Examples of organic acids that can be used to activate the magnetic nanoparticles not functionalized by phospholipid molecules include acetic acid, lactic acid, propanoic acid and/or butanoic acid.

Step d of the process for preparing the biocompatible oily ferrofluid consists of surface functionalizing the magnetic nanoparticles of the colloidal sol by at least one phospholipid. For this, the colloidal sol is contacted with the phospholipid(s). For example, a solution of phospholipid(s) can be added to the colloidal sol with mechanical stirring and at ambient pressure. Any known technique for colloidal sol formation can be used.

The solution of phospholipid(s) used can comprise one or more phospholipids, according to the functionalization of the magnetic nanoparticles sought, and at least one solvent in which the phospholipid(s) are soluble, hereinafter called solvent S3. Advantageously, the phospholipids bear a phosphate function in the salified form, especially with an alkaline cation such as a sodium atom, to promote the coordination/complexation of the phosphate function of the polar head of said phospholipid on the magnetic nanoparticles.

Phospholipids are generally in the salt form for stability purposes.

The solvent S3 can be an organic solvent. Preferably, this solvent S3 is volatile.

Examples of suitable solvents S3 include chloroform, alone or in mixture with methanol (in a ratio of 2:1 v:v, for example), diethyl ether, heptane, dichloromethane or isopropanol.

Preferably this solvent (or mixture of solvents) S3 is the same as the volatile organic solvent S2 used as the dispersive medium in the colloidal sol of magnetic nanoparticles.

The phospholipids used in this phospholipid solution are those detailed for the phospholipid molecules functionalizing the magnetic nanoparticles of the biocompatible oily ferrofluid according to the invention.

The phospholipid content in the magnetic nanoparticle dispersion in organic solvents S2 and S3 depends on the total surface developed by the magnetic nanoparticles used, the phospholipid used and its developed surface and the fatty acid esters used. Advantageously, the phospholipid content in the reaction medium is comprised between 0.005 and 10% by mass, preferably from 0.05 to 2.5% by mass relative to the total mass of the colloidal sol.

According to a first embodiment, the phospholipid molecules are present in excess relative to the surface developed by the non-functionalized magnetic nanoparticles, preferably corresponding to 1000% of the quantity necessary to form a monolayer saturated with phospholipids. Indeed, an excess of phospholipids shifts the reaction equilibrium and increases the chemisorption kinetics of phospholipids at the surface of the magnetic nanoparticles. Such an excess is advantageous when this functionalization reaction is not kinetically favorable, for example when the polar phosphate head of the phospholipid molecules used is sterically hindered. The reaction time for phospholipid molecules contained in the colloidal sol is then the parameter to consider depending on the excess of phospholipid molecules. The reaction is stopped by the addition of a solvent causing nanoparticle flocculation and precipitation of the excess phospholipid molecules, such as ethanol, for example. Thus, for example, for a 2000% excess of phospholipids with a hindered polar head such as phosphatidylserine, the chemisorption time necessary for the dispersion of the magnetic nanoparticles in the oils described in the context of the invention is chosen in the interval from 5 to 30 minutes, preferably from 6 to 10 minutes, and better still from 7 to 8 minutes. According to this embodiment, the process comprises a subsequent step of eliminating the excess phospholipid molecules, for example by successive washes, preferably with a mixture of organic solvents, such as an ethanol/chloroform mixture (3:1) as well as a final step of eliminating the chloroform with ethanol.

According to a second embodiment, the quantity of phospholipid molecules used is present in deficit with respect to the developed surface of the non-functionalized magnetic nanoparticles, preferably in a percentage ranging from 38% to 100% of the quantity necessary to form a monolayer saturated with phospholipid(s), and in particular ranging from 38% to less than 100%. Indeed, a deficit of phospholipid molecules allows controlling the functionalization rate of the magnetic nanoparticles by the phospholipid molecules. Such a deficit is advantageous when the functionalization reaction is kinetically very favorable, for example when the phospholipid used has an —O(O)P(OH)O⁻, M⁺, polar head, with M being an alkali metal atom, preferably a sodium atom.

At the end of the functionalization step d, the percentage of coverage and/or the surface density of the functionalization of the magnetic nanoparticles is such as described for the functionalized magnetic nanoparticles of the biocompatible oily ferrofluid according to the invention. The conditions for the process according to the invention are adjusted by the skilled person to have such coverage percentages and/or such surface densities of functionalization.

Once the functionalization of the magnetic nanoparticles has been completed, the dispersive medium of the colloidal sol and almost all of the solvent S3 of the phospholipid solution used are eliminated (step e), for example by evaporation under a flow of inert gas (nitrogen or argon) to prevent phospholipid oxidation, in order to obtain aggregates of magnetic nanoparticles functionalized by phospholipid molecules.

Finally, these functionalized nanoparticles are dispersed in an oil phase comprising at least one fatty acid ester (step e). Any traces of solvents S2 and S3 and mainly ethanol are evaporated under reduced pressure, for example in the rotary evaporator at 80° C. for an effective 30 min. The oil phase and the fatty acid ester are such as described for the biocompatible oily ferrofluid according to the invention. This dispersion in the colloidal form in the oil phase occurs either spontaneously at ambient temperature, or with heating to a temperature ranging from 30 to 80° C. Most often, this temperature is determined according to the grafting rate and the phospholipid molecules, oil and fatty acid ester employed. Such heating can occur in step e) or in a later heating to obtain the colloidal dispersion.

Use of a Biocompatible Oily Ferrofluid According to the Invention.

The invention also concerns the use of a biocompatible oily ferrofluid according to the invention or obtained according to the preparation process according to the invention.

The biocompatible oily ferrofluid according to the invention, or obtained according to the process of the invention, can be used as a medicament, in particular for cancer treatment, and especially for the treatment of benign or malignant nodules or solid tumours, by magnetic hyperthermia.

Advantageously, this medicament to treat cancer is made up, in a first embodiment, solely of the biocompatible oily ferrofluid. Alternatively, according to a second, non-preferred embodiment, the medicament comprises the biocompatible oily ferrofluid as well as lipophilic cancer treatment agents.

In order to treat a cancer patient, an intratumoral administration of biocompatible oily ferrofluid is carried out using any technique known to the skilled person, then an external alternating magnetic field is applied in order to induce heating of the magnetic nanoparticles comprised in the biocompatible oily ferrofluid. This localized heating dissipates throughout the volume of the oil phase of the oily ferrofluid, thus enabling the destruction of the tumour cells.

According to a particular embodiment, this heating is accompanied by the release of a therapeutic agent contained in a heat-sensitive support, or can be used for the activation of a heat-activatable therapeutic agent or to induce the expression of a gene under transcriptional control of a heat-sensitive promoter or to exert a synergistic effect with another therapeutic agent co-administered independently in the context of chemotherapy and/or radiation therapy protocols.

Typically, the alternating magnetic field applied is situated in the range from 5 to 25 kA/m, while the frequency is situated in the range from 100 to 750 kHz. The duration of the field/frequency pair applied depends on the thermal dose per unit of volume dissipated for an oily ferrofluid of a given iron oxide mass concentration as a function of the tumour volume to be treated. These protocols for treatment by hyperthermia are calibrated for different field/frequency pairs according to the type of treatment sought, which requires different thermal doses, such as thermoablation, release of a therapeutic agent, activation of a heat-activatable therapeutic agent or the induction of expression of a gene under the transcriptional control of a heat-sensitive promoter.

According to a first embodiment, the biocompatible oily ferrofluid is used as the sole medicament in this treatment.

According to a second embodiment, especially when the oily ferrofluid does not comprise a chemotherapy medicament, the biocompatible oily ferrofluid is used in combination with another chemotherapy medicament, such as paclitaxel, docetaxel or carmustine, preferably contained in the magnetic oil. A co-injection of an injectable formulation containing heat-sensitive active ingredients such as Thermodox® can also be considered.

The biocompatible oily ferrofluid according to the invention, or obtained according to the process according to the invention, can also be used as a contrast agent for medical imaging, such as MRI, near-infrared (NIR) fluorescence imaging, and local fiber approaches by endoscopy.

In the case of use in fluorescence imaging, the oily ferrofluid administered to the patient preferably comprises lipophilic fluorophores.

The invention also concerns an imaging method for the entire body or a part of the body of an individual comprising a step of obtaining one or more images of said entire body or part of the entire body by a medical imaging technique, said entire body or said part of the entire body comprising the contrast medium comprising the biocompatible oily ferrofluid.

Advantageously, the oily ferrofluids according to the invention are dispersed in colloidal form, at least at the temperature which is reached during the induction treatment: the magnetic nanoparticles are then well dispersed, do not form aggregates and do not flocculate over time.

Even more advantageously, the ferrofluids are stable, in particular for at least 24 hours, advantageously for at least 1 month, at a temperature belonging to the range 20-40° C. at atmospheric pressure and, especially at room temperature (20° C.) or physiological temperature (37° C.). It is possible to differentiate short-term stability (duration less than or equal to 24 h) which will be evaluated under ambient air and long-term stability (duration greater than 24 h). The long-term stability can especially be evaluated by keeping the ferrofluid under inert atmosphere such as argon or nitrogen and protected from light and at the temperature at which the stability must be evaluated, especially at 20° C. or 37° C.

In the case where the ferrofluids according to the invention correspond to a colloidal dispersion, only at a temperature greater than ambient temperature, especially at a temperature belonging to the range from 20 to 40° C., it might be necessary to keep the ferrofluids in thermostated containers or chambers or even to heat them before use to a temperature allowing a colloidal dispersion to be obtained. They could also be administered by injection using thermostated infusions or by implementing heating kits or devices to maintain the ferrofluid at a temperature in the range of 20 to 40° C., at which the colloidal stability is satisfactory.

Nanoemulsion Comprising a Biocompatible Oily Ferrofluid According to the Invention.

The invention also concerns a biocompatible oily ferrofluid nanoemulsion according to the invention, or obtained according to the process for preparing an oily ferrofluid according to the invention, as shown schematically in FIG. 1.

“Nanoemulsion” means an emulsion whose dispersed phase droplets in the continuous phase are of nanometric size, i.e., between 100 nm and less than 300 nm

In the context of the invention, the nanoemulsion can be a simple oil-in-water or water-in-oil type emulsion, or a multiple emulsion of the water-in-oil-in-water type. Preferably, the nanoemulsion according to the invention is an oil-in-water emulsion.

According to a first embodiment, the lipophilic phase of the nanoemulsion is made up solely of the biocompatible oily ferrofluid.

According to a second embodiment, the lipophilic phase of the nanoemulsion comprises the biocompatible oily ferrofluid in combination with one or more other lipophilic compounds chosen especially among oils, lipophilic fluorophores, perfluorocarbons or lipophilic active ingredients such as for example paclitaxel, docetaxel, etoposide, carmustine.

Moreover, in order to improve their pharmacokinetics, the interphase of magnetic lipid droplets may contain one or more PEGylated lipids (phospholipids) and/or targeting ligands (antibodies, peptides, aptamers).

The lipophilic compounds excluding active cancer treatment ingredients that can be used in the nanoemulsion according to the invention are preferably biocompatible and do not generate any toxicity.

Any biocompatible oil known in the state of the art can be used. Examples of oils that can be used in the context of the invention as another lipid compound of the nanoemulsion include soybean oil, olive oil, sesame oil, cottonseed oil, poppyseed oil, copra oil, coconut oil, flaxseed oil, sunflower oil, fatty acid triglyceride oils such as Miglyol 812N® or Labrafac® WL 1349, fatty acid propylene glycol oils such as Miglyol 840® or Labrafac® PG and fish oils.

When the nanoemulsion comprises an oil or a mixture of oils in addition to the biocompatible oily ferrofluid, these oils are present in a content belonging, in general, to the range of from 10 to 35% by mass, preferably from 20 to 30% by mass relative to the total mass of the nanoemulsion.

Lipophilic fluorophores that can be used in the context of the invention include fluorescent analogues of phospholipids and sphingomyelin, cyanines such as indocyanine green (ICG) and lipophilic carbocyanines.

When the nanoemulsion comprises lipophilic fluorophores, these oils are preferably present in a content belonging to the range of 0.0001 to 0.02% by mass, preferably from 0.001 to 0.01% by mass relative to the total mass of the nanoemulsion.

Targeting ligands are known to allow recognizing the biological target by molecular interaction, and thus improve the specificity of the mixture administered. Examples of targeting ligands include agents based on amino acids such as folic acid, sugars such as mannose or FDG (fluorodeoxyglucose commonly used in PET imaging), peptide sequences such as the cyclic RGD peptide directed against integrins or (Tyr3)—octreotate (TATE) with high affinity for somatostatin type 2 receptors, synthetic compounds such as raclopride acting as an antagonist of dopamine D2 receptors, antibodies of different formats such as those from camelids (nanobodies) or fragments of recombinant antibodies selected by phage display screening methods such as scFv directed against PSMA or anti PD1/PDX1 used in immunotherapies or aptamers selected by SELEX screening methods directed against membrane receptors of cancer cells.

The aqueous phase of the nanoemulsion according to the invention is typically made up of water, optionally in combination with one or more solvents miscible with water such as ethanol and propylene glycol. The aqueous phase of the nanoemulsion can also comprise salts (sodium or potassium chlorides) or buffers.

Moreover, the nanoemulsion can comprise one or more surfactants, also called dispersion agents, preferably biocompatible. These surfactants and dispersion agents are then present at the aqueous phase/lipophilic phase interface of the nanoemulsion.

Examples of surfactants and dispersion agents which may be suitable within the scope of the invention include egg or soy lecithins, bile acids such as sodium deoxycholate, polyoxyethylenated castor oil, polysorbate 20, polysorbate 40, polysorbate 60, polysorbate 80, sorbitan monolaurate (Span® 20, Span® 40, Span® 60, and/or Span® 80), poloxamers or PEG block copolymers.

When these agents are present, the surfactant and/or dispersion agent content typically belongs to the range from 1 to 5% by mass, preferably from 1.8 to 3.8% by mass, relative to the total mass of the nanoemulsion.

Advantageously, the nanoemulsions according to the invention are stable at ambient temperature (20° C.) at atmospheric pressure. In particular, the nanoemulsions according to the invention are stable for at least 24 hours, preferably for at least 6 months at ambient temperature (20° C.) and at atmospheric pressure.

In the context of the invention, the colloidal stability of the nanoemulsion is determined by evaluation of the particle-size distribution by dynamic light scattering (DLS), as detailed in the examples.

Preparation Process for a Nanoemulsion According to the Invention

The invention also concerns a process for preparing a nanoemulsion according to the invention.

Such a process for preparing a nanoemulsion comprises the following successive steps:

i—providing a lipophilic phase comprising a biocompatible oily ferrofluid according to the invention or obtained according to the process for preparing a biocompatible oily ferrofluid according to the invention,
ii—providing an aqueous phase,
iii—mixing the lipophilic phase and the aqueous phase to form a nanoemulsion.

In particular, preferably, the mixture of step iii will be done at a temperature where the oily ferrofluid according to the invention is found in the form of a colloidal dispersion. The lipophilic phase comprising the biocompatible oily ferrofluid according to the invention could, also, be heated at such a temperature before being mixed with the aqueous phase.

The process for preparing the nanoemulsion according to the invention can comprise an optional step i2, after step i and before step iii, of bringing the biocompatible oily ferrofluid into contact with one or more compounds chosen from oils, lipophilic fluorophores, targeting ligands, lipophilic surfactants, pharmacophores, such as described above, in order to obtain the lipophilic phase of the nanoemulsion. This optional step can be performed by mixing at ambient temperature and pressure according to any technique known to the skilled person.

At step ii, the aqueous phase can comprise one or more surfactants, such as described above, when they are present.

At step iii, the mixture of the aqueous and lipophilic phases is conventionally carried out at high speed using any technique known to the skilled person. For example, the lipophilic and aqueous phases, optionally previously heated, in particular to a temperature in the range from 60 to 70° C., are first mixed at high speed in order to form a coarse emulsion, then homogenized using a sonicator or by high-pressure homogenization.

The aqueous phase, the lipophilic phase and the surfactant(s) optionally present are such as described for the nanoemulsion according to the invention.

Use of a Nanoemulsion According to the Invention

The invention also concerns the use of a nanoemulsion according to the invention or of a nanoemulsion obtained according to the process of the invention.

A nanoemulsion according to the invention can be used as a medicament, in particular in the treatment of cancer by magnetic-induced hyperthermia. For this, a systemic administration of the nanoemulsion is performed, followed by the application of an external alternating magnetic field. In this case, the magnetic field is applied at the moment when the accumulation of product in the tumour has reached its maximum. This wait time between the injection and the application of the alternating magnetic field is estimated by optical imaging (fiber or not), by MRI or by ultrasound (for formulations based on perfluorocarbons). For the case of an intratumoral administration, the magnetic field can be applied following the injection. The characteristics of magnetic induction (field/frequency pair values) and duration of application) for the nanoemulsions can be adjusted according to the desired thermal doses. The heating of the magnetic oil droplets can be accompanied by the release of a therapeutic agent or can be used for the activation of a heat-activatable therapeutic agent or to induce the expression of a gene under the transcriptional control of a heat-sensitive promoter or exert a synergistic effect with another therapeutic agent co-administered independently in the context of chemotherapy and/or radiation therapy protocols.

According to a first embodiment, the nanoemulsion is used as the sole medicament in this treatment.

According to a second embodiment, especially when the nanoemulsion does not comprise a chemotherapy medicament, the nanoemulsion is used in combination with another chemotherapy medicament of lipophilic nature, such as taxanes (paclitaxel and docetaxel) or carmustine. A co-injection of an injectable formulation containing heat-sensitive active ingredients such as Thermodox® can also be considered.

The volumes and concentrations of the oily ferrofluids administered as well as the number of injections and the time between each injection, as applicable, especially depend on the tumour treated (location, volume) and the patient (age, physical condition) and are determined by the practitioner.

The nanoemulsion according to the invention can also be used as a contrast product for medical imaging, such as MRI, ultrasound or near-infrared (NIR) fluorescence imaging, and local fiber approaches by endoscopy.

In the case of use in fluorescence imaging, the nanoemulsion administered to the patient preferably comprises lipophilic fluorophores.

The invention also concerns an imaging method for the entire body or a part of the body of an individual comprising a step of obtaining one or more images of said entire body or part of the entire body by a medical imaging technique, said entire body or said part of the entire body comprising the contrast product comprising the emulsion.

Kits

The invention also concerns a kit comprising a container in which is placed the oily ferrofluid according to the invention or the nanoemulsion according to the invention. The kit can also comprise magnetic nanoparticles surface functionalized by molecules of one or more phospholipids such as described according to the invention, alone in one container and optionally the oil phase containing at least one fatty acid ester intended to make up the oily ferrofluid in another separate container. The ferrofluid or nanoemulsion can be reconstituted from the kit just before its use or administration. The kit can also comprise conditions for use and terms for the field/frequency pairs applicable and the corresponding thermal dose dissipated per unit of volume of ferrofluid of a given concentration. It should be noted that the iron concentration is also certified for indication purposes.

### EXAMPLES

Materials and Methods:

A) Phospholipids Tested:

The phospholipids used to prepare the oily ferrofluids in the examples below are:

1,2-dioleoyl-sn-glycero-3-phosphoethanolamine (DOPE) of Chem. formula 1 below:

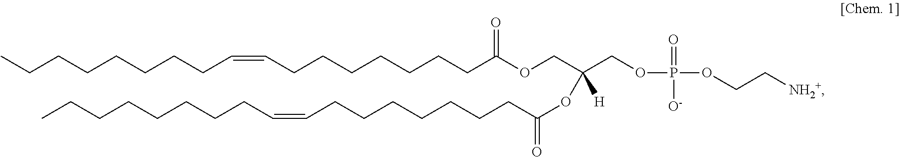

1,2-dioleoyl-sn-glycero-3-phosphatidic acid (DOPA), of Chem. formula 2 below (in the sodium salt form):

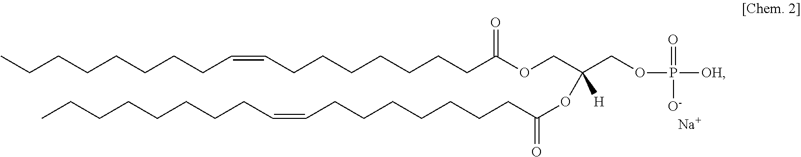 [SMILES: CCCCCCCC/C=C\CCCCCCCC(=O)OC[C@H](COP(=O)([O-])O)OC(=O)CCCCCCC/C=C\CCCCCCCC ; [Na+]]

1,2-distearoyl-sn-glycero-3-phosphatidic acid (DSPA) of Chem. formula 3 below (in the sodium salt form):

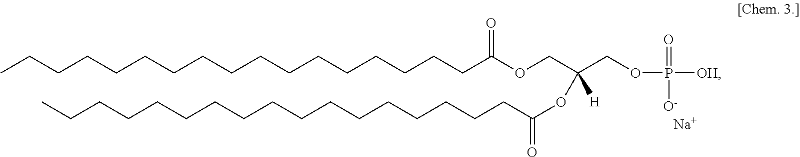 [SMILES: CCCCCCCCCCCCCCCCCC(=O)OC[C@H](COP(=O)([O-])O)OC(=O)CCCCCCCCCCCCCCCCC ; [Na+]]

and

1,2-dioleoyl-sn-glycero-3-phospho-L-serine (DOPS) of Chem. formula 4 below (in the sodium salt form):

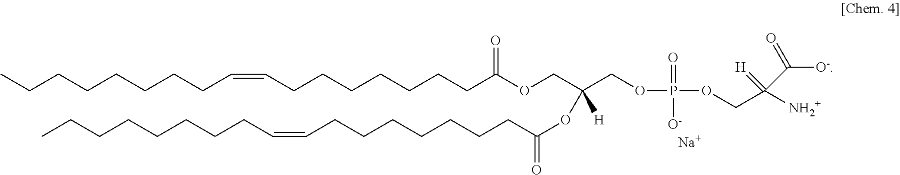

B) Composition of the Oil Phases:

The oil phases used in the examples are mixtures of fatty acid triglycerides or fatty acid propylene glycols: Miglyol 812N® and Miglyol 840®, respectively. Table 1 below indicates the fatty acids making up the fatty acid triglycerides or propylene glycols of making up these oils (the percentages are mass percentages relative to the total weight of the oil).

TABLE 1

| | Miglyol 812 | Miglyol 840 |
|---|---|---|
| Caprylic acid | 50-56% | 65-80% |
| Capric acid | 30-45% | 20-35% |
| Lauric acid | 2% | 3% |

C) Iron Determination:

The iron content is determined by visible UV spectroscopy by dissolving nanoparticles of iron oxide in a 5 M solution of hydrochloric acid (HCl).After complete dissolution of the nanoparticles in 5 M HCl, the absorbance at 350 nm is measured to determine the iron concentration. The iron concentration is determined according to the formula: A^{350 nm}=ε.l.[Fe]. The molar extinction coefficient ε equals 2960 L⁻¹.mol.cm⁻¹.

D) Size of the Nanoparticles:

D1) Measurement by Dynamic Light Scattering (DLS)

The hydrodynamic radius of the nanoparticles is determined by dynamic light scattering using a Cordouan Vasco device equipped with a laser with a wavelength of 658 nm and at an angle of 135°; the data are acquired for a duration of 60 sec.

D2) Measurement by X-Ray Diffraction (XRD)

The size of the crystallites is determined by XRD by using the Scherrer formula Math. 1 below, where F is the mean diameter of the crystallites, λ the wavelength of the X-rays (λ(Cu Kα)=1.5406 Å), β the width at mid-height of the most intense peak and Θ the Bragg angle:

$$Γ = \frac{0.9 λ}{β * cos ( θ )} . \qquad [ Math . 1 ]$$

D3) Measurement by Transmission Electron Microscopy (TEM)

The average elementary size of the nanoparticles is determined by transmission electron microscopy by measuring the size of 200 nanoparticles using image processing software (ImageJ Rasband, W. S., ImageJ, U. S. National Institutes of Health, Bethesda, Md., USA, https://imagej.nih.gov/ij/, 1997-2019), obtained with a Philips CM120 microscope operating at 120 kV and equipped with an Ultra scan USC1000 camera (2k×2k).

E) Surface Characterization:

E1) Thermogravimetric Analysis (TGA)

Approximately 10 mg of previously dried nanoparticles under reduced pressure at 70° C. are put in a platinum crucible, and the analysis is performed using a Tag2400 thermobalance from Setaram. The sample is heated to 600° C. under air with a ramP of 5° C./min.

E2) Diffuse Reflection Infrared Spectroscopy

The diffuse reflection infrared spectroscopy (DRIFT) is done with a Bruker IFS Equinox 55 spectrometer. The functionalized nanoparticles are oven-dried at 70° C. after the washing steps. Then the nanoparticles are ground in anhydrous KBr (3% by mass).

E3) Determination of the Surface Functionalization Densities and the Theoretical (Also Called Nominal) and Actual Coverage Rates

The surface developed by the phospholipid molecule sprayed over a surface depends on the volume occupied by the polar head in comparison with that of the lipid chains. In the case of phospholipids, the bulk volume of the two parts (polar and hydrophobic) is equivalent so that a lipid is a cylinder rather than a cone. It is this particular feature that allows them to assemble themselves into vesicular membranes rather than micelles. The area bulk values of phospholipids are generally between 60 and 65 Å² depending on the environment in which they are found, alone or in a mixture, the number of unsaturations, interactions with a solid surface (supported lipid membrane) or in a liquid medium (vesicle) and different physicochemical parameters such as temperature. The reference value of a phospholipid molecule comprising an —O(O)P(OH)O⁻, Na⁺ phosphatidyl head ((as is the case of DOPA and DSPA) is 62 Å² corresponding to the molecular crowding within a saturated monolayer contained in a lipid membrane. This molecular surface density value is the one found for lipid membranes (phospholipid bilayers) by considering a single monolayer. Obviously, there are slight differences when the OH function is substituted. Thus for dioleoyl phosphatidylserine, the developed molecular surface area is around 65 Å²/molecule and for dioleoylphosphatidylethanolamine is generally measured between 60 and 65 Å². It is therefore chosen below at 62 Å². This value of 62 Å² is used, in the context of the invention, for calculating the percentages of coverage of the surface of the nanoparticles and for calculating the surface functionalization densities in phospholipid molecules, regardless of the phospholipid envisaged.

The “nominal or theoretical coverage rate” in phospholipid is the molar percentage represented by the quantity of lipid involved in relation to the quantity necessary to form a monolayer, calculated from the average size of the nanoparticles measured by TEM and the surface of reference of the polar head of the phospholipid used. When the nanoparticles are spheroids, it is considered that the average size of the nanoparticles measured by TEM is taken as the diameter to evaluate the average surface developed by the particles. For nanoflowers, given their surface roughness, their developed surface was estimated by repeated coatings of silica of different thickness by sol-gel route (Stöber synthesis route). This method thus made it possible to estimate the surface area 15% greater than that of a smooth sphere of the same diameter.

TGA measurements make it possible to determine the quantity of phospholipid molecules actually present on the nanoparticles per surface unit from the total mass loss measured between 200° C. and 600° C., from the molar mass of the organic residues decomposed during complete combustion relative to the surface developed by the nanoparticles involved. The grafting yield represents the ratio between the quantity of phospholipid molecules chemisorbed on the surface of the nanoparticles and the nominal quantity involved during the reaction.

F) Synthesis of Iron Oxide Nanoparticles

F1) Synthesis of the FF1 Nanoparticles by Alkaline Coprecipitation

A mass of 31.41 g of FeCl₂.4H₂O (0.158 mol) dissolved in 170 mL 1.5 M hydrochloric acid is poured into a beaker containing 85.4 g of FeCl₃.6H₂O (0.316 mol) dissolved in 3.5 L of water (initial stoichiometric ratio Fe²⁺/Fe³⁺=0.5). Under high mechanical stirring, 300 mL of an ammonia solution (28-30% m/m) are added quickly. The medium is left under stirring for 15 min. The magnetite nanoparticle flocculates are decanted by means of a permanent magnet and then the supernatant is eliminated. After two successive steps of washing with water, the surface of the nanoparticles is oxidized by adding a volume of 200 mL of 2 M HNO₃ then left under stirring for 15 min. After decantation and elimination of the supernatant, the core of the nanoparticles is oxidized into maghemite by adding 600 mL of a 0.33 M iron nitrate solution. The reaction medium is boiled for 30 min. After decantation and elimination of the supernatant, 200 mL of 2 M nitric acid are added. The flocculate is then magnetically decanted and washed 3 times with acetone in order to eliminate the excess acid. The flocculate is finally peptized in 200 mL of water. After evaporation of the excess acetone, the ferrofluid is brought to a volume of 1 L with water. Subsequently, the dispersion of maghemite nanoparticles is called FF1. At the end of synthesis, the ferrofluid has a mass concentration of iron oxide of 69 g/L and develops a surface area per unit volume of 11316 m²/L. The average elementary size of the spheroid nanoparticles thus obtained determined by TEM is 7.5 nm±2 nm, and 6.9 nm when it is measured by XRD. The hydrodynamic radius measured is D_{H}=38 nm.

F2) Synthesis of the FF2 Nanoparticles by Polyol

A mass of 1.082 g of FeCl₂.4H₂O (5.44 mmol) and of 0.398 g of FeCl₃.6H₂O (1.47 mmol) are dissolved in 80 g of a mixture of diethylene glycol (DEG) and N-methyldiethanolamine (NMDEA) (ratio 1:1, v/v). This solution is mixed for 1 h under nitrogen flow with stirring until complete dissolution of the precursors. A mass of 0.64 g of NaOH (16 mmol) is dissolved in 40 g of a NMDEA/DEG mixture (1: 1, v/v) under nitrogen flow. The NaOH solution is added to the solution containing the precursors and the mixture is heated to 220° C., at 2° C./min, for 4 h. The nanoparticles obtained are magnetically sedimented and washed with an ethanol/ethyl acetate (1:1, v/v) mixture three times to eliminate the organic and inorganic impurities. A wash with 10% nitric acid is performed. 8.25 g of Fe₃(NO₃)₃.9H₂O (20.4 mmol) are solubilized in 20 mL of water and the solution is added to the nanoparticles. The nanoparticle dispersion is heated at 80° C. for 45 minutes to obtain maghemite. After decantation and elimination of the supernatant, the nanoparticles are washed with 10% nitric acid, then with acetone and finally with diethyl ether. Finally, the nanoparticles are redispersed in water. Subsequently, the dispersion of maghemite nanoparticles is called FF2. At the end of synthesis, the ferrofluid has a mass concentration of iron oxide of 20 g/L and develops a surface of 1574 m²/L. The average elementary size of the nanoparticles of nanoflower morphology thus obtained is measured at 15.4 nm±3 nm by the TEM method and 16 nm by XRD. The hydrodynamic radius measured is D_{H}=26 nm.

Synthesis of the FF3 Nanoparticles by Polyol

The FF3 nanoparticles are synthesized under the same operating conditions as those described for obtaining the FF2 nanoparticles except that the temperature increase to 220° C., at 2° C./min, for 4 h takes place this time under thermally insulated reaction conditions. To do this, glass wool was placed around the flask in contact with the walls of the reactor in the open air and above the mantle heater in order to limit heat loss, thus allowing better kinetic control of the temperature ramp. The average elementary size of the nanoparticles of nanoflower morphology obtained by the TEM method is 18.5 nm±3.1 nm.

G) Colloidal Stability in Oily Dispersions

The colloidal stability in the dispersions is observed as a function of temperature in a first approximation, visually and then by measuring the transmittance at 800 nm with a VARIAN Cary 500 spectrophotometer equipped with a temperature regulator. The quality of the nanoparticle dispersions is also checked by DLS (method D1). When “+” appears in the tables of the examples, this means that the colloidal character is obtained by dispersing the nanoparticles in the oil phase at the corresponding temperature and that this colloidal character is still present at least 24 hours after it is obtained, the time elapsed at least between the preparation of the dispersion and the measurement of the transmittance.

### Example 1: Dispersion of the FF1, FF2 and FF3 Nanoparticles in Chloroform

The purpose of this example is to transfer the nanoparticles of the aqueous ferrofluids FF1, FF2 and FF3 into chloroform without resorting to surfactants in order to be able to perform the phospholipid grafting of by chemisorption. To do so, a volume of 1 mL of an ammonia solution (28-30%, m/m) is added to 35.4 mL of FF1 at 69 g/L in γ—Fe₂O₃. The nanoparticle flocculates are decanted by means of a permanent magnet and then the supernatant is eliminated. The aggregated nanoparticles are washed twice with water. Then the aggregated nanoparticles are washed five times with ethanol. Finally, a volume of 80 mL of chloroform is added to the nanoparticles and the nanoparticles are redispersed in an ultrasound bath for 3 minutes. The final concentration is 30.5 g/L in γ—Fe₂O₃ in chloroform. The protocol is identical for ferrofluids FF2 and FF3.

### Example 2: Preparation of Biocompatible Oily Ferrofluids Resulting from the Functionalization, in the Presence of Acid, of Magnetic Nanoparticles by Phospholipid Molecules Presenting an Unhindered Polar Head

### Example 2.1: Dispersions of Nanoparticles Functionalized with Monounsaturated Phospholipid Molecules

### Example 2.1.1: Dispersion in Miglyol M840® of Magnetic Nanoparticles of Ferrofluid FF1 Functionalized by DOPA

In this example, a dispersion of magnetic nanoparticles of FF1 functionalized by DOPA in Miglyol M840® is prepared. The reference area bulk of a phospholipid molecule is 62 Å² corresponding to the molecular crowding within a saturated monolayer contained in a lipid membrane. The functionalization protocol for nanoparticles with a nominal coverage rate (corresponding to the quantity of lipid involved compared to the quantity necessary to form a monolayer) of 55% (i.e. 0.89 molecule/nm²) compared to the quantity necessary to form a DOPA monolayer is as follows: A volume of 16.4 mL of magnetic nanoparticles ([γ—Fe₂O₃]=30.5 g/L, developed surface=82 m²) dispersed in chloroform, prepared in Example 1, are diluted in a volume of 47.7 mL of an acetic acid solution in chloroform (1:9, v/v). To this dispersion, a volume of 3.51 mL of DOPA (25 mg/mL in chloroform, 45.2 m²) is added under vortex. After 14 h of reaction at 4° C., ethanol is added into the medium until the nanoparticles flocculate and the whole is magnetically decanted. The magnetic flocculate is washed with 3 volumes of 100 mL of chloroform/ethanol mixture (1:3, v/v) then with 2 volumes of 100 mL of ethanol. At the end of the last wash, the nanoparticles are dried under nitrogen flow and a volume of 15 mL of Miglyol M840® is added to the nanoparticles. The nanoparticles are dispersed from 35° C. in oil after heating in a water bath.

Likewise, a range of nominal DOPA coverage rates set between 20% and 150% relative to the monolayer, corresponding to a theoretical grafting density comprised between 0.33 DOPA/nm² and 2.42 DOPA/nm², can be performed by working at a constant DOPA concentration (1.25 mg/mL). For certain nominal density variables, the nanoparticles spontaneously disperse at ambient temperature (20° C.).

The nominal DOPA coverage rate is calculated according to the equation Math. 2 below.

$$coverage rate = 100 * \frac{{N}^{PL} * 0.62}{4 π * {N}^{NP} * {R}^{2}} . \qquad [ Math . 2 ]$$

where N^{PL} is the number of phospholipid molecules, N^{NP} is the number of nanoparticles and R is the nanoparticle radius in nm.

The stability of the nanoparticle dispersions derived from FF1 functionalized by DOPA in Miglyol M840® was evaluated at different temperatures and different coverage rates as summarized in Table 3 below. The criteria designating the quality of the dispersions are: −=non-dispersible (turbid medium), +=colloidal sol stable over time (clear dispersion). These screening criteria can be conveniently evaluated by visual observations in good agreement with transmittance measurements at 800 nm. TGA measurements made it possible to determine the actual DOPA quantity per unit of surface and the grafting yield. The nominal and actual DOPA surface densities and the colloidal stability of nanoparticles in M840® at 20° C. and 37° C. are summarized in Table 2 below.

TABLE 2

| | | | | | | | | | |
|---|---|---|---|---|---|---|---|---|---|
| Nominal coverage | 20% | 30% | 38% | 44% | 55% | 70% | 90% | 100% | 150% |
| rate | | | | | | | | | |
| Actual coverage | 19.9% | 28.0% | 29.8% | 34.8% | 35.4% | 36.0% | 45.3% | 49.1% | 75.8% |
| rate | | | | | | | | | |
| Quantity of DOPA | 0.33 | 0.48 | 0.61 | 0.71 | 0.89 | 1.13 | 1.45 | 1.61 | 2.42 |
| added per unit of | | | | | | | | | |
| surface d_{DOPA/nm}² | | | | | | | | | |
| Actual quantity of | 0.32 | 0.45 | 0.48 | 0.56 | 0.57 | 0.58 | 0.73 | 0.79 | 1.22 |
| DOPA per unit of | | | | | | | | | |
| surface d_{DOPA/nm}² | | | | | | | | | |
| Stability at T = 20° C. | − | − | − | + | + | + | + | + | − |
| Stability at T = 37° C. | − | − | + | + | + | + | + | + | + |
| Stability at T = 70° C. | + | + | + | + | + | + | + | + | + |
| D_{H} (nm)* | 489 | 450 | 268 | 112 | 58 | 49 | 42 | 53 | 293 |
| Transmittance at | 41 | 44 | 53 | 70 | 75 | 84 | 82 | 83 | 40 |
| 800 nm (%) at | | | | | | | | | |
| 20° C.** | | | | | | | | | |

*D_{H}: Hydrodynamic diameter measured by DLS at 20° C.
**The transmittance values were determined by dispersions at 0.2 g/L in Fe₂O₃.

The chemisorption of DOPA on the nanoparticles is confirmed by DRIFT (FIG. 2) with the presence of a band between 1070 cm⁻¹ and 1170 cm⁻¹ and 1200 cm⁻¹ respectively corresponding to the vibrations of the P—OH and P—O bonds. The TGA analysis also confirms the chemisorption of DOPA on the nanoparticles. TGA analyses make it possible to determine actual grafting yields of between 49% and 97.5% over the entire range, and from 51% to 80% over the colloidal stability domain (FIG. 3). These yields are advantageous since they allow a minimized use of phospholipid molecules.

### Example 2.1.2: Dispersion in Miglyol 812N® of Magnetic Nanoparticles of Ferrofluid FF1 Functionalized by DOPA

The protocol used is the same as the one described in Example 2.1.1 except that the modified nanoparticles are redispersed in Miglyol 812N®. The range is created under the same conditions, i.e., a constant DOPA concentration (1.25 mg/mL).

The stability of the nanoparticle dispersions derived from FF1 functionalized by DOPA in Miglyol 812N® was evaluated at different temperatures and different nominal coverage rates as summarized in Table 3 below. The criteria designating the quality of the dispersions are: −=non-dispersible (turbid medium), +=colloidal sol stable over time (clear dispersion).

TABLE 3

| | | | | | | | | | | |
|---|---|---|---|---|---|---|---|---|---|---|
| Nominal coverage rate | 20% | 25% | 35% | 44% | 50% | 60% | 70% | 80% | 90% | 100% |
| Theoretical d_{DOPA/nm}² | 0.33 | 0.40 | 0.56 | 0.71 | 0.81 | 0.96 | 1.12 | 1.29 | 1.44 | 1.55 |
| Actual d_{DOPA/nm}² | 0.32 | 0.39 | − | 0.56 | 0.57 | 0.59 | 0.60 | 0.67 | 0.73 | 0.79 |
| Dispersibility at T = 20° C. | − | − | − | − | + | + | + | − | − | − |
| Dispersibility at T = 35° C. | − | − | − | + | + | + | + | + | − | − |
| Dispersibility at T = 70° C. | − | − | + | + | + | + | + | + | + | + |

### Example 2.1.3: Dispersion in Miglyol M840® of Magnetic Nanoparticles of Ferrofluid FF2 Functionalized by DOPA

The DOPA chemisorption protocol is identical to the one described in Example 2.1.1 except that the nanoparticles of ferrofluid FF2 are used.

The stability of the nanoparticle dispersions derived from FF2 functionalized by DOPA in Miglyol M840N® was evaluated at different temperatures and different nominal coverage rates as summarized in Table 4 below. The criteria designating the quality of the dispersions are: −=non-dispersible (turbid medium),

+=colloidal sol stable over time (clear dispersion). The hydrodynamic diameter (D_{H}) values of the nanoparticles of ferrofluid FF2 were measured by externalized DLS (VASCO-FLEX, Cordouan Technologies) for different DOPA grafting rates, at 60° C. under induction by application of an external alternating magnetic field (755 kHz, 10.2 kA.m⁻¹).

TABLE 4

| | | | | | | | | | |
|---|---|---|---|---|---|---|---|---|---|
| Nominal coverage rate | 22% | 31% | 39% | 44% | 53% | 61% | 71% | 79% | 82% |
| Theoretical d_{DOPA/nm}² | 0.34 | 0.48 | 0.60 | 0.69 | 0.82 | 0.95 | 1.10 | 1.22 | 1.32 |
| Dispersibility at T = 20° C. | − | − | − | − | − | − | − | − | − |
| Dispersibility at T = 35° C. | − | − | − | − | − | − | − | − | − |
| Dispersibility at T = 60° C. | − | − | − | − | − | + | + | + | + |
| D_{H} (60° C., in nm) | 342 | 259 | 238 | 322 | 261 | 53 | 39 | 45 | 40 |

### Example 2.1.4: Dispersion in Miglyol M840® of Magnetic Nanoparticles of Ferrofluid FF3 Functionalized by DOPA

The DOPA chemisorption protocol for dispersing magnetic nanoparticles in Miglyol M840® is identical to that described in Example 2.1.1 except that the nanoparticles of ferrofluid FF3 are used by applying a nominal coverage rate of 1.10 molecules/nm². Two dispersions of mass concentrations equal to 5 g/L and 300 g/L are prepared for SAR (specific absorption rate) measurements and in vivo thermoablation experiments on tumours carried by mice (Example 6).

### Example 2.2: Preparation of a Dispersion of Nanoparticles Functionalized with Phospholipid Molecules Having a Substituted Polar Head: Dispersion in Miglyol M840® of Magnetic Nanoparticles of Ferrofluid FF1 Functionalized by DOPE

The protocol used is the same as the one described in Example 2.1.1 except for the nature of the phospholipid employed. The range is created under the same conditions, i.e., a constant DOPE concentration (1.25 mg/mL).

The stability of the nanoparticle dispersions derived from FF1 functionalized by DOPE in Miglyol M840® was evaluated. The results are summarized in Table 5 below. These results are given as a function of temperature and coverage rate. The criteria designating the quality of the dispersions are: −=non-dispersible (turbid medium),

+=colloidal sol stable over time (clear dispersion).

TABLE 5

| Nominal coverage rate | 24% | 48% | 52% | 61% |
|---|---|---|---|---|
| Theoretical d_{DOPA/nm}_{²} | 0.39 | 0.77 | 0.84 | 0.98 |
| Dispersibility at T = 25° C. | − | − | − | − |
| Dispersibility at T = 35° C. | − | − | + | − |
| Dispersibility at T = 70° C. | − | + | + | + |

At 52% of the coverage rate, the functionalized nanoparticles are dispersible from 35° C. At 70° C., the stability range is widened between 48 and 61% of the nominal coverage rate. The presence of a substituent on the polar head affects the chemisorption kinetics relative to those of DOPA and reduces the colloidal stability range in surface composition and temperature.

### Example 2.3: Preparation of a Dispersion of Nanoparticles Functionalized with Saturated Phospholipid Molecules Having a Non-Substituted Polar Head: Dispersion in Miglyol M840® of Magnetic Nanoparticles of Ferrofluid FF1 Functionalized by DSPA

The protocol used is the same as the one described in Example 2.1.1 except for the nature of the phospholipid employed. The range is created under the same conditions, i.e., a constant DSPA concentration (1.25 mg/mL).

The stability of the nanoparticles derived from FF1 functionalized by DSPA in Miglyol M840N® was evaluated at different temperatures and different coverage rates as summarized in Table 6 below. The criteria designating the quality of the dispersions are: −=non-dispersible (turbid medium),

+=colloidal sol stable over time (clear dispersion).

TABLE 6

| Nominal coverage rate | 10% | 24% | 48% | 58% | 61% |
|---|---|---|---|---|---|
| Dispersibility at T = 25° C. | − | − | − | − | − |
| Dispersibility at T = 35° C. | − | + | − | − | − |
| Dispersibility at T = 70° C. | − | + | + | + | + |
| Dispersion temperature | − | 35° C. | 45° C. | 52° C. | 58° C. |

The dispersion temperature of the magnetic nanoparticles, i.e. the temperature above which the dispersion of the magnetic nanoparticles is clear, depends on the amount of DSPA used. The dispersion temperature of the magnetic nanoparticles was determined by transmission measurements at 800 nm by temperature, according to the protocol detailed above.

The nanoparticles FF1 functionalized by DSPA are dispersible at temperatures above ambient in biocompatible oils, from 35° C. at a nominal coverage rate of 24%. The temperature of dispersion in Miglyol M840® increases with the coverage rate in phospholipid molecules.

### Example 3: Preparation of Biocompatible Oily Ferrofluids Resulting from the Functionalization of Magnetic Nanoparticles by Phospholipid Molecules in the Absence of Acid

### Example 3.1: Preparation of Biocompatible Oily Ferrofluids by Surface Functionalization of Nanoparticles Using Phospholipid Molecules with Substituted Phosphate Heads: Case of Magnetic Nanoparticles Derived from FF1 Modified with DOPS in Miglyol M840®

The steric hindrance offered by certain polar heads of substituted phospholipids has the effect of slowing down the kinetics of chemisorption at the iron oxide surface. However, these kinetics can be accelerated by acting on the increase in the phospholipid concentration, i.e., by introducing said phospholipid molecules in excess relative to a monolayer of phospholipid molecules. In this case, the reaction becomes the key parameter to control the number of phospholipid molecules grafted by nanoparticles.

A volume of 14.2 mL of DOPS (25 mg/mL in chloroform) is added under vortex at 1.64 mL of magnetic nanoparticles FF1 ([Fe₂O₃]=30.5 g/L) dispersed in chloroform. The chemisorption reaction is stopped, at different reaction times, by adding ethanol to inhibit the reaction inducing the flocculation of the nanoparticles and the precipitation of the excess phospholipid molecules. The suspension is then decanted by a magnet. The flocculate is washed three times with 50 mL of ethanol. During the last washing, the nanoparticles are dried under nitrogen flow and a volume of 15 mL of Miglyol M840® is added to the nanoparticles. The nanoparticles spontaneously disperse in the oil.

The stability of the nanoparticle dispersions derived from FF1 functionalized by DOPS in Miglyol M840N® was evaluated after different reaction times leading to different nominal coverage rates as summarized in Table 7 below. The criteria designating the quality of the dispersions are: −=non-dispersible (turbid medium),

+=colloidal sol stable over time (clear dispersion).

TABLE 7

| | | | | | | |
|---|---|---|---|---|---|---|
| Incubation time (min) | 1 | 7 | 20 | 30 | 60 | 90 |
| Dispersibility at T = 25° C. | − | + | − | − | − | − |

TGA makes it possible to follow the DOPS chemisorption kinetics over time. Table 8 and FIG. 4 show the variations in actual grafting density of functionalized nanoparticles with 2000% excess DOPS either after 7 min of reaction, washing and drying, or after 30 min of reaction, washing and drying. FIG. 4 represents the thermograms of nanoparticles functionalized with an excess of 20% of DOPS at 7 min and at 30 min of reaction, thus obtained.

TABLE 8

| Time (min) | ΔM % | d_{DOPA/nm}_{²} |
|---|---|---|
| 7 min | 19.1 | 0.87 |
| 30 min | 22.5 | 1.02 |

### Example 3.2: Preparation of Biocompatible Oily Ferrofluids by Surface Functionalization of Nanoparticles Using Phospholipid Molecules with Non-Substituted Phosphate Heads: Case of Magnetic Nanoparticles Derived from FF1 Modified with DOPA in Miglyol M840®

The protocol for preparing nanoparticles with a phospholipid deficit of 50% (i.e., _{DOPA/nm2}=0.81 DOPA.nm⁻²) is as follows: a volume of 16.4 mL of magnetic nanoparticles FF1 ([γ—Fe₂O₃]=30.5 g/L) dispersed in chloroform are diluted in 44 mL of chloroform. To this dispersion, a volume of 3.18 mL of DOPA (25 mg/mL in chloroform) is added under vortex. After 14 h of reaction at 4° C., ethanol is added until the nanoparticles flocculate and the whole is magnetically decanted. The flocculate is washed with a chloroform/ethanol mixture (1:3, v/v) and then with ethanol. During the last washing, the nanoparticles are dried under nitrogen flow and a volume of 15 mL of Miglyol M840® is added to the nanoparticles. The nanoparticles spontaneously disperse in the oil.

Likewise, a range of nominal DOPA coverage rates located between 5% and 96% in deficit relative to the molecular crowding within a monolayer of a lipid membrane, corresponding to a coverage density comprised between 0.08 DOPA/nm² and 1.55 DOPA/nm², can be performed by working at a constant DOPA concentration (1.25 mg/mL).

The stability of the nanoparticles derived from FF1 functionalized by DOPA in Miglyol M840N® without addition of acid during the reaction was evaluated at different temperatures and different nominal coverage rates as summarized in Table 9 below. The criteria designating the quality of the dispersions are: −=non-dispersible (turbid medium),

+=colloidal sol stable over time (clear dispersion).

TABLE 9

| | | | | | | | | | | |
|---|---|---|---|---|---|---|---|---|---|---|
| Nominal coverage rate | 20% | 25% | 35% | 44% | 50% | 60% | 70% | 80% | 89% | 96% |
| Theoretical d_{DOPA/nm}² | 0.33 | 0.40 | 0.56 | 0.71 | 0.81 | 0.96 | 1.12 | 1.29 | 1.44 | 1.55 |
| Dispersibility at T = 25° C. | − | − | − | − | − | + | + | + | − | − |
| Dispersibility at T = 35° C. | − | − | − | + | + | + | + | + | − | − |
| Dispersibility at T = 70° C. | − | + | + | + | + | + | + | + | + | + |

### Example 4: Comparison of Oily and Aqueous Ferrofluids Derived from FF1, FF2 and FF3

Dispersions of nanoparticles with an iron oxide mass concentration of 5 g/kg of solvent in the biocompatible oil phases or in an aqueous medium are prepared to measure and compare their heating under magnetic induction. The temperature of the samples is measured by an optical fiber (OTG-M420, Opsen™) and the temperature of the samples is set at 37° C.

Alternating magnetic fields are generated by two different devices, in order to access different field/frequency pairs. The first device is DM3 from nB nanoScale Biomagnetics offering the following conditions: 473.5 kHz and 13.36 kA. m⁻¹, 344.5 kHz and 16.23 kA. m⁻¹, 217 kHz and 20.09 kA. m⁻¹, and 146 kHz, 21.96 kA. m⁻¹.

A second device, consisting of an induction coil supplied by a Minimax Junio™ 1TS 3.5 kW generator provides access to an additional condition: 755 kHz, 10.2 kA.m⁻¹.

Measuring the heating rate under magnetic induction allows determining the thermal power of the nanoparticles per unit of mass of magnetic liquid, called specific adsorption rate (SAR). The SAR is determined according to the formula Math. 3 below:

$$SAR = \frac{{m}_{s}}{{m}_{NP}} {C}_{P} \frac{Δ T}{Δ t} . \qquad [ Math . 3 ]$$

where C_{P} represents the heat capacity of the dispersion medium in J.(g.K)⁻¹ (C_{water}=4.18 J.g⁻¹.K⁻¹ and Cₒᵢₗ=2, J.g⁻¹.K⁻¹)m_{S} the solvent mass, m_{NP} the nanoparticle mass and ΔT/Δt represents the slope at the origin (37° C.) of the tangent of the temperature evolution curve as a function of time.

The temperature profiles of the magnetic aqueous dispersions FF1, FF2 and FF3 of iron oxide mass concentration equal to 5 g/L, compared to the profiles obtained with the nanoparticles obtained in Examples 2.1.1, 2.1.3 and 2.1.4, derived, respectively, from FF1, FF2 and FF3 functionalized by DOPA with theoretical coverage densities respectively of 0.81, 1.29 and 1.10 molecules/nm² and dispersed in Miglyol M840® under magnetic induction are represented in FIGS. 5A, 5B and 5C, respectively for two field/frequency pairs ((FIG. 5A) 755 kHz, 10.2 kA/m, (FIG. 5B) 473.5 kHz, 13.36 kA/m, (FIG. 5C) 473.5 kHz, 13.36 kA/m).

In 10 seconds, the temperature of the aqueous ferrofluid FF1 increases by 0.12° C. while for nanoparticles derived from FF1 functionalized by DOPA dispersed in Miglyol M840®, the increase is 0.5° C., corresponding to a temperature gain of more than a factor of 4. Over an induction period of 100 seconds, this same gain is measured, i.e. a temperature increase of more than 4.8° C. in the oil phase and only 1.2° C. in the aqueous phase. Concerning FF2, in 10 seconds, the temperature of the aqueous dispersion FF2 increases by 0.8° C., while, for the FF2 dispersions functionalized by DOPA and dispersed in Miglyol M840®, the temperature increase is 5.8 times higher, i.e. 5.2° C. An even higher increase is observed for FF3 dispersions in oil: in 10 seconds, a temperature increase of 3° C. is measured for the aqueous ferrofluid, while the temperature increase is 12 times greater, i.e., 36° C., for the oily ferrofluid. The oily dispersion of FF3 therefore has clearly superior heating properties relative to the aqueous dispersion of FF3 with a very rapid temperature increase.

The SAR values measured in both media (aqueous and oil) for the three types of nanoparticles FF1, FF2 and FF3 are listed in Table 10 below:

TABLE 10

| | Field/frequency | |
|---|---|---|
| | pair applied | SAR (W/g) |
| FF1 H₂O | 755 kHz, 10.2 kA.m⁻¹ | 10 |
| FF1 M840 | | 20 |
| FF1 H₂O | 473.5 kHz, 13.36 kA.m⁻¹ | 12.1 |
| FF2 H₂O | | 83.6 |
| FF2 M840 | | 220 |
| FF3 H₂O | | 295 |
| FF3 M840 | | 2805 |

The SAR values at 37° C. for the FF1 dispersion under induction at 755 kHz, 10.2 kA/m are doubled between the aqueous and oily media. The SAR values at 37° C. for the two types of magnetic nanoparticles with nanoflower morphology resulting from dispersions FF2 and FF3 subjected to induction at 473.5 kHz, 13.36 kA/m are multiplied by a factor of 2.6 and 8.8, respectively, between the aqueous and oily medium.

The stability of these oily ferrofluids subjected to several magnetic induction cycles has also been evaluated.

The oily and aqueous dispersions of FF2 were subjected to 4 successive heating/cooling cycles (ΔT>10K, i.e., from 37° C. to more than 47° C.) under magnetic induction for 2 different field/frequency pairs (2 cycles at 344.5 kHz, 16.23 kA.m⁻¹ followed by 2 cycles at 473.5 kHz, 13.36 kA.m⁻¹).

The oily and aqueous dispersions of FF3 were subjected to 2 successive heating/cooling cycles (ΔT>40K, i.e., from 37° C. to more than 77° C.) for two magnetic induction cycles for a field/frequency pair of 473.5 kHz, 13.36 kA.m⁻¹.

FIGS. 6A, 6B and 6C represent, respectively, the temperature profiles of the dispersions of nanoparticles FF2 and FF3 dispersed in water (5 g/kg) and of the same nanoparticles functionalized by DOPA (for respective theoretical coverage densities of 1.29 and 1.10 molecules/nm², acidic condition, Examples 2.1.3 and 2.1.4) in Miglyol M840 (5 g/kg) during the application of the alternating magnetic field. In FIG. 6A, 2 cycles of heating under induction at 473.5 kHz, 13.36 kA.m⁻¹ and cooling (T₀=37° C.) are performed. In FIG. 6B, 2 cycles of heating under induction at 344.5 kHz, 16.23 kA.m⁻¹ and cooling (T₀=37° C.) are performed. In FIG. 6C, 2 cycles of heating under induction at 473.5 kHz, 13.36 kA.m⁻¹ and cooling (T₀=37° C.) are performed.

The SAR values measured for these aqueous and oily dispersions are detailed in Table 11 below.

TABLE 11

| | | SAR 1^{st} | SAR 2^{nd} | ΔT (° C.) | ΔT (° C.) |
|---|---|---|---|---|---|
| | Field/frequency | cycle | cycle | in 10s | in 10s |
| | pair applied | (W/g) | (W/g) | 1^{st} cycle | 2^{nd} cycle |
| FF2 H₂O | 344 kHz, | 92 | 100.3 | 1.1 | 1.2 |
| FF2 M840 | 16.23 kA.m⁻¹ | 284 | 300 | 7.1 | 7.5 |
| FF2 H₂O | 473.5 kHz, | 83.6 | 83.6 | 1.0 | 1.0 |
| FF2 M840 | 13.36 kA.m⁻¹ | 220 | 252 | 5.5 | 6.3 |
| FF3 H₂O | | 291 | 298 | 3.4 | 3.5 |
| FF3 M840 | | 2411 | 2800 | 39.1 | 43.4 |

The SAR values at 37° C. of the magnetic nanoparticles derived from the FF2 dispersions are multiplied by a factor of 3, respectively, between the aqueous and oily media for both field/frequency conditions 344 kHz, 16.23 kA.m⁻¹ and 473.5 kHz, 13.36 kA.m⁻¹. For an induction time of 10 seconds, the temperature of the aqueous dispersion FF2 increases by approximately 1° C. while for the same nanoparticles derived from FF2 functionalized with DOPA and dispersed in Miglyol M840®, the increase in temperature is approximately 6 to 7 times higher.

The SAR values at 37° C. of the magnetic nanoparticles derived from the FF3 dispersions are multiplied by a factor of 8.3 to 9.4 between the aqueous and oily media for field/frequency conditions 473.5 kHz, 13.36 kA.m⁻¹. For an induction time of 10 seconds, the temperature of the aqueous dispersion FF3 increases by approximately 3.5° C. while for the same nanoparticles derived from FF3 functionalized with DOPA and dispersed in Miglyol M840®, the increase in temperature is approximately 11 to 12 times higher. The oily ferrofluids can therefore potentially make it possible to drastically reduce the treatment time under induction.

Performing several heating cycles by induction demonstrates that the dispersions withstand the local temperature increase of the nanoparticles with no changes in the properties of the nanoparticles and the oil phase. There is no degradation of the phospholipid molecules on the surface of the nanoparticles. This property can prove interesting from the perspective of meeting the need to multiply magnetic-induced hyperthermia treatment sequences.

The SAR values were also measured by varying the dispersion volume of nanoparticles FF3 (5 g/Kg) from 500 μL to 1 μL, in water and in oil (Miglyol M840®) from the temperature profiles represented in FIGS. 7A, 7B and 7C.

The thermogenic power of the dispersions as well as the temperature plateau value decrease with the volume of the dispersions due to the thermal losses which become increasingly predominant with the increase in the surface area to volume ratio. For oily dispersions, the thermal losses are much lower relative to the aqueous ferrofluid due to their weaker thermal conductivity and the absence of endothermal phenomena such as evaporation. For volumes of 200 and 500 μL, the heating power is still as high, 9 times greater than that of aqueous dispersions, and no temperature plateau can be observed for these measurement conditions. Temperature thresholds of 85° C. and 75° C. are reached fairly quickly (2-3 minutes), respectively, for the volumes of 50 and 10 μL due to their still very high SAR values. The plateau values and especially the SAR values remain much higher, by a factor of approximately 5 to 10, than those of the aqueous dispersions of FF3. The FF3 in Miglyol M840® dispersion thus shows very high SAR values even for small volumes up to 10 μL.

The SAR value for a volume of 1 μL is measured by directly depositing the ferrofluid at the tip of the sensor. The initial temperature is then that of the room (T0=25° C.). The SAR of 65 W/g is only measurable for the dispersion of FF3 in Miglyol M840®. The evaporation of water manifested by a decrease in the temperature of the medium of 7° C. is too rapid to measure the SAR of the aqueous dispersion FF3 for this volume.

FIG. 8 represents the kinetic temperature profile for a volume of 1 μL of dispersion FF3 dispersed in Miglyol M840® used for the animal experiments of thermoablation of tumours by magnetic hyperthermia presented in Example 6. The dispersion of mass concentration in iron oxide Fe₂O₃ is 300 g/L (T0=25° C., ambient room temperature) has a kinetic heating profile showing a temperature increase of 75° C. in 20 s for a SAR value of 53 W/g. This dispersion concentrated at 300 g/L allows very rapid local temperature increases, even for very small volumes, of the order of a μL, which can contribute to improving the precision of the treatment in the context of thermoablations of low volume tumours.

### Example 5: Preparation of Nanoemulsions from the Oily Ferrofluid

The magnetic nanoparticles FF1 functionalized by DOPA obtained at point 2.1.1 fora coverage density of 1.12 molecules/nm² are dispersed in the oil phase made up of Miglyol 840. The oil phase containing the dispersed iron oxide nanoparticles is either used alone directly and then makes up 100% of the oil phase of the nanoemulsion, or can be diluted beforehand in Miglyol 840 to make up ⅓ or ⅔ of the total mass of the lipophilic phase of the nanoemulsion.

The nanoemulsions are prepared by optionally mixing the lipophilic phase (Miglyol 840) with the oily ferrofluid in order to obtain a homogeneous mixture in which in which egg lecithin E80 is dispersed when hot (70° C.). The aqueous phase previously heated to the same temperature is mixed with the co-surfactant (polysorbate 80). The emulsion and homogenization is obtained in a single step by phase inversion using a sonicator for 10 minutes. After obtaining the nanoemulsion, this remains stable after the temperature is lowered, especially to a temperature of 20° C.

Examples of compositions of nanoemulsions 1 to 5 are detailed in Tables 12 to 16 below, in which the percentages are mass percentages relative to the total weight of the nanoemulsion.

TABLE 12

| Nanoemulsion 1 | % |
|---|---|
| Ferrofluid FF1-DOPA (60 mg/mL) in | 20 |
| dispersion in Miglyol 840 | |
| Miglyol 840 | 0 |
| Egg lecithin E80 | 1.2 |
| Polysorbate 80 | 2 |
| Water | qs 100 |

TABLE 13

| Nanoemulsion 2 | % |
|---|---|
| Ferrofluid FF1-DOPA (60 mg/mL) in | 30 |
| dispersion in Miglyol 840 | |
| Miglyol 840 | 0 |
| Egg lecithin E80 | 1.8 |
| Polysorbate 80 | 2 |
| Water | qs 100 |

TABLE 14

| Nanoemulsion 3 | % |
|---|---|
| Ferrofluid FF1-DOPA (60 mg/mL) in | 35 |
| dispersion in Miglyol 840 | |
| Miglyol 840 | 0 |
| Egg lecithin E80 | 2.1 |
| Polysorbate 80 | 2 |
| Water | qs 100 |

TABLE 15

| Nanoemulsion 4 | % |
|---|---|
| Ferrofluid FF1-DOPA (60 mg/mL) in | 20 |
| dispersion in Miglyol 840 | |
| Miglyol 840 | 10 |
| Egg lecithin E80 | 1.8 |
| Polysorbate 80 | 2 |
| Water | qs 100 |

TABLE 16

| Nanoemulsion 5 | % |
|---|---|
| Ferrofluid FF1-DOPA (60 mg/mL) in | 10 |
| dispersion in Miglyol 840 | |
| Miglyol 840 | 20 |
| Egg lecithin E80 | 1.8 |
| Polysorbate 80 | 2 |
| Water | qs 100 |

The zeta potential (ZP) measurements are performed by diluting the sample to 1/1000th in deionized water. The ZP value is determined by electrophoresis and detection by laser Doppler using a Zetasizer Nano ZS apparatus (Malvern Instruments SA, Worcestershire, UK). The particle size characteristics (mean hydrodynamic diameter, polydispersity index (PDI) and zeta potential (ZP) of the examples are given in the following Table 17:

TABLE 17

| | Nanoemulsion | Nanoemulsion | Nanoemulsion | Nanoemulsion | Nanoemulsion |
|---|---|---|---|---|---|
| | 1 | 2 | 3 | 4 | 5 |
| Mean diameter | 161.2 ± 0.6 | 187.6 ± 2.4 | 188.3 ± 2.3 | 183.7 ± 5.0 | 191.5 ± 0.4 |
| (nm) | | | | | |
| PDI | 0.112 | 0.119 | 0.121 | 0.125 | 0.101 |
| PZ (mV) | 31.8 ± 2.4 | 33.8 ± 0.7 | 40 ± 2.5 | 32.2 ± 0.8 | 36.7 ± 0.3 |

The thermogenic power of the nanoemulsions 1 and 4 presenting the same iron oxide concentration of the ferrofluid FF1 (CFe₂O₃=12 g/L, T₀=37° C.) under induction at 473.5 KHz, 13.36 kA/m is compared to FIG. 9.

At equal ferrofluid concentration but with a larger oil phase percentage, the heating is improved (comparison emulsions 1 and 4).

### Example 6: Thermoablation of Subcutaneous Tumours by Magnetic Hyperthermia

The efficacy of the oily ferrofluid according to Example 2.1.4, composed of magnetic nanoparticles FF3 functionalized by DOPA in Miglyol M840®, for thermoablation by magnetic hyperthermia of tumours in mice were evaluated.

In order to follow the distribution of the product in the tumour medium, a lipophilic fluorophore emitting in the near infrared was previously incorporated into the oily ferrofluid: 1,1′-dioctadecyl-3,3,3′,3′-tetramethylindotricarbocyanine iodide (DiR). The maximum emission of this fluorophore is situated at 780 nm.

The mice tested are B6 albino mice (B6N-Tyrc-Brd/BrdCrCrl) bearing a tumour implanted subcutaneously RM1-CMV-LucF in a paw. The tumour volumes were estimated from the l and w dimensions measured with the digital caliper and calculated from Feldman's formula: Volume=π/6×f×(lx·w)^{3/2} with f=1.58 for female mice. The different volumes were then estimated around 170±20 mm³ depending on the specimen.

Given the tumour volumes, the experiments were carried out by performing intratumoral microinjections of oily ferrofluid with an iron oxide concentration of 300 μg/μL. The oily ferrofluids were injected directly into the tumour. The injections were performed using a 10 μL Hamilton syringe equipped with a beveled needle (26 gauge), under isoflurane anaesthesia then the mice are placed in a heated bed under the induction coils. Only one induction treatment at 473.5 kHz and 13.36 kA.m⁻¹ for 15 minutes was performed for each mouse.

In a first experiment, an intratumoral injection of 2 μL of oily ferrofluid FF3 (300 μg/μL in iron oxide, i.e., a mass of 600 μg) is performed at a depth of 2.5 mm on a new specimen separated into two times 1 μL with a one-minute wait between the two injections (performed at the same injection point). At the end of the last injection, a wait time of one minute is applied again before withdrawing the needle. The thermal dose dissipated by the nanoparticles in the tumoral volume (Q_{V}) can, in first approximation, be calculated according to the formula: Q_{V}=m_{P} (Fe₂O₃)×SAR(1 μL)×t/Vₜᵤₘₒᵤᵣ. Under these operating conditions, the thermal dose Q_{V}=0.17 J/mm³. The bioluminescence and fluorescence images were produced following the injection before the induction treatment and 24 h after. As illustrated in FIG. 10A, one area of thermoablation can be clearly observed at the center of the tumour. During the application of the alternating magnetic field, the temperature difference measured by the thermal camera was 3° C. Tumour growth, measured by integration of bioluminescence 24 h later, is slowed. The ex vivo fluorescent images after tumour resection show the location of the ferrofluid injection area. The ex vivo bioluminescence image indicates a loss of viability of cancer cells at the site where the ferrofluid was injected (FIG. 10B).

In a second experiment, multiple injections of three times 1 μL of oily ferrofluid FF3 (300 μg/μL, i.e., a mass of 900 μg) were performed at a depth of 2.5 mm, distributed over three distinct sites of the tumour (FIG. 11A). A wait time of one minute was applied before withdrawing the needle for each injection. Under these operating conditions, the thermal dose Q_{V}=0.25 J/mm³. 24 h after the application of the alternating magnetic field (ΔT≈20° C.), the tumour ablation areas in the vicinity of the ferrofluid injection areas can be easily distinguished. The ex vivo measurements confirm the partial ablation of the tumour at the areas treated (FIG. 11B).

1. A biocompatible oily ferrofluid comprising iron-oxide based magnetic nanoparticles and an oil phase comprising at least one fatty acid ester, wherein the magnetic nanoparticles are surface functionalized by molecules of one or more phospholipids.

2. The oily ferrofluid according to claim 1, comprising iron-oxide based magnetic nanoparticles and an oil phase comprising at least one fatty acid ester, said iron-oxide based magnetic nanoparticles forming a colloidal dispersion in said oil phase from a temperature belonging to the range from 20 to 80° C., wherein the magnetic nanoparticles are surface functionalized by molecules of one or more phospholipids which do not completely cover the surface of the iron-oxide based magnetic nanoparticles and, in particular, which ensure a coverage rate of the surface of the iron-oxide based magnetic nanoparticles such that the fatty acid ester(s) present in the oil phase have access to the surface of the iron-oxide based magnetic nanoparticles.

3. The oily ferrofluid according to claim 1, wherein the oily ferrofluid is free of water and/or free of surfactant.

4. The oily ferrofluid according to claim 1, wherein the phospholipid molecules ensure a coverage of 19 to 76%, preferably 29 to 76%, and preferentially 34 to 50%, of the surface of iron-oxide based magnetic nanoparticles.

5. The oily ferrofluid according to claim 1, wherein the surface density of grafting of the magnetic nanoparticles by phospholipid(s) molecules belongs to the range from 0.32 molecule/nm² to 1.22 molecules/nm², preferably 0.48 molecule/nm² to 1.22 molecules/nm², and preferentially 0.56 molecule/nm² to 0.79 molecule/nm².

6. The oily ferrofluid according to claim 1, wherein the phospholipid(s) contain at least one fatty chain, preferably two fatty chains, in particular C6-C30, preferably C8-C24, or even C10-C22, and in particular, C18 saturated or mono- or polyunsaturated, branched or preferably linear hydrocarbon chains.

7. The oily ferrofluid according to claim 1, wherein the oil phase comprises at 70% by mass of fatty acid esters, preferably the oil phase comprises 80% to 95% by mass of fatty acid esters, relative to the total mass of the oil phase.

8. The oily ferrofluid according to claim 1, wherein the fatty acid ester(s) of the oil phase are chosen from C6-C12, preferably C6-C10, saturated fatty acid triglycerides and C6-C12, preferably C6-C10, saturated fatty acid propylene glycols, used alone or as a mixture.

9. The oily ferrofluid according to claim 1, wherein the magnetic nanoparticle content belongs to the range from 0.01% to 50% by mass, preferably from 0.1% to 10% by mass, relative to the total mass of the oily ferrofluid.

10. The oily ferrofluid according to claim 1, wherein the magnetic nanoparticles are in the shape of a spheroid, a polyhedron such as nanocubes, bipyramids or nanostars, a wafer, a nanorod, a nanodisk or a nanoflower.

11. The oily ferrofluid according to claim 1, wherein the phospholipid(s) have an —O(O)P(OH)O⁻ polar head and are preferably chosen from salts of 1,2-dioleoyl-sn-glycero-3-phosphatidic acid and 1,2-distearoyl-sn-glycero-3-phosphatidic acid.

12. The oily ferrofluid according to claim 1, further comprising a lipophilic active ingredient, in particular chosen from cancer treatment drugs, such as Paclitaxel, Docetaxel or Carmustine.

13. A process for preparing the oily ferrofluid according to claim 1, comprising the following successive steps:
a—providing an aqueous dispersion of iron-oxide based magnetic nanoparticles in an aqueous solvent which can be water or a mixture of water/solvent(s) miscible with water,
b—eliminating the aqueous solvent from the aqueous dispersion of magnetic nanoparticles,
c—obtaining a colloidal sol of magnetic nanoparticles by addition of solvent or a mixture of volatile organic solvents (S2),
d—surface functionalizing said magnetic nanoparticles of the colloidal sol with molecules of at least one phospholipid,
e—eliminating said volatile organic solvent(s) (S2) and dispersing the functionalized magnetic nanoparticles in an oil phase comprising at least one fatty acid ester.

14. The process according to claim 13, further comprising a step c2, after step c and before step d, of adding acid.

15. A medicament comprising the oily ferrofluid according to claim 1.

16. An oil-in-water nanoemulsion comprising from 10% to 30% by mass of the oily ferrofluid according to claim 1, an aqueous phase and a surfactant.

17. A contrast product comprising the biocompatible oily ferrofluid according to claim 1.

18. A biocompatible oily ferrofluid comprising iron-oxide based magnetic nanoparticles and an oil phase comprising at least one fatty acid ester, wherein the magnetic nanoparticles are surface functionalized by molecules of one or more phospholipids, or the biocompatible oily ferrofluid obtained according to the process of claim 13, for its use in cancer treatment by magnetic-induced hyperthermia.

19. A medicament comprising the oily ferrofluid obtained according to the process of claim 13.

20. An oil-in-water nanoemulsion comprising from 10% to 30% by mass of the oily ferrofluid obtained according to the process of claim 13, an aqueous phase and a surfactant.
